(19) United States
(12) Patent Application Publication
(10) Pub. No.: US 2025/0170327 A1
(43) Pub. Date: May 29, 2025

(54) SYSTEM AND METHOD FOR A CONSTANT FORCE SYRINGE PUMP ACCOMODATING SYRINGES OF DIFFERENT SIZES

(71) Applicant: KORU Medical Systems, Inc., Mahwah, NJ (US)

(72) Inventors: BRIAN CHRISTOPHER CASE, Lake Villa, IL (US); ERIK MARIUS KARLSEN, Vernon, NJ (US); STEVEN ANDREW WATTS, San Diego, CA (US); PETER ANDREW SMITH, Zebulon, NC (US); GABRIEL DORAN JEFFER, Raleigh, NC (US); DEREK TEIG ROBERTS, Chicago, IL (US); THOMAS RALPH BLACKBURN, III, Holly Springs, NC (US); GABRIELLA Anne Colonna, Ridgewood, NJ (US); ROBERT GREGORY HOFF, Boonton, NJ (US); WESLEY ROBERT HOWELL, Ringwood, NJ (US); PAUL JOSEPH TARSIA, Ringwood, NJ (US)

(73) Assignee: KORU Medical Systems, Inc., Mahwah, NJ (US)

(21) Appl. No.: 18/961,267

(22) Filed: Nov. 26, 2024

Related U.S. Application Data
(60) Provisional application No. 63/603,176, filed on Nov. 28, 2023.

(51) Int. Cl. A61M 5/142

(52) U.S. Cl. CPC A61M 5/142

(57) ABSTRACT

Provided is a constant force pump accommodating syringes of different sizes. The system is provided by an enclosure having a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base; a sizer structured and arranged to determine the size of the chamber; an adjustable driver structured and arranged to provide a constant force between the pusher and the puller, the constant force adjustably selected by the size of the chamber. An associated method of use is provided as well.

### CROSS REFERENCE TO RELATED APPLICATIONS

This application claims the benefit under 35 U.S.C. § 119 (e) of U.S. Provisional Application No. 63/526,176 filed Nov. 28, 2023, and entitled SYSTEM AND METHOD FORA CONSTANT FORCE SYRINGE PUMP ACCOMMODATING SYRINGES OF DIFFERENT SIZES, the disclosure of which is incorporated herein by reference.

### FIELD OF THE INVENTION

The present invention relates generally to mechanical syringe pumps for delivering a liquid medicant to a patient, and more specifically to a mechanical constant force syringe pump advantageously adapting to the different size of each syringe to ensure the proper constant force is applied for dispensation of the liquid medicant to the patient in accordance with the prescribed rate of medicant flow for the presently engaged syringe.

### BACKGROUND

Infusion systems for the delivery of liquid pharmaceuticals are widely used and relied upon by patients and care givers alike to provide infusion therapy.

One form of infusion therapy is Immune Globulin (Ig) therapy, and it is frequently used to improve the quality of life for patients with conditions such as Primary Immune Deficiency (PID), Secondary Immune Deficiency (SID), Chronic Inflammatory Demyelinating Polyradiculoneuropathy (CIPD), and Severe Combined Immunodeficiency (SCID). Historically, Ig therapy has been administered intravenously (IVIg) every 3-4 weeks, which can result in inconsistent serum levels and burdensome infusion experiences for patients.

Subcutaneous Ig therapy (SCIg) is a more convenient option that allows for consistent serum levels and can be done at home. Subcutaneous administrations have been shown to maintain a more constant IgG blood level which provides the patient with a consistent quality of life, and less breakthrough infections. Additionally, infusion to subcutaneous tissues is often preferred over intravenous delivery which is often accompanied by flu-like symptoms following such intravenous delivery. However, SCIg has its own challenges, such as the need for patients to prepare and administer the infusions themselves.

At least one challenge relates to the pump system used for subcutaneous Ig therapy. There are essentially two varieties of home pump systems-constant pressure and constant flow. With a constant flow rate system, the pressure is increased in response to any flow restriction no matter if such a restriction is the build-up of pressure in the patient's tissues or an element of the delivery system. This can result in an administration of the liquid at an unsafe pressure. As such, the patient may suffer a wide range of symptoms, including, but not limited to, anaphylaxis, overdose, histamine reactions, morbidity, and mortality.

In contrast, a constant pressure pump generates a safe and limited constant pressure. If there is a pinch in the tubing causing blockage in the infusion system or blockage in the patient's body (such as by saturation of the tissues), such a blockage results in resistance to the flow and affects the flow rate, not the pressure, i.e., the flow rate decreases as the pressure increases. As such, constant pressure pump systems have been found to be safer and are often more financially acceptable to users.

But there is also the issue of dosage. Frequently dosage is pre-determined and provided in pre-filled syringes that may be disposed within a constant pressure pump. However, each person is different in size, shape, and tissue makeup among many other factors. Bodyweight itself could necessitate a prescription for a 17 g (85 ml) dose for Ig therapy for one person, but only 10 g for another person. However, syringes come in a variety of sizes as well-such as 5, 10, 20 and 50 ml, with each varying in length as well as diameter.

If the medication is provided in a syringe other than the one the constant pressure pump is designed for, the patient or caregiver must transfer the liquid medicant from the pre-filled syringe into a new and sterile pump compliant syringe. Such a transfer process is not without issues and introduces the opportunity for problems, such as but not limited to complete transfer, contamination, and general stress and concern.

As 10 ml syringe has a different length and diameter from that of a 50 ml syringe, a different amount of force should be applied to the syringe to generate the needed constant pressure to provide the same expected and intended initial flow rate while also providing the safeguard of the flow rate from the pump decreasing as resistance pressure increases due to the infusion system itself or issues within the patient.

More simply stated, if a 10 ml syringe was disposed in a pump designed for a 50 ml syringe, the constant force applied to the 10 ml syringe would be far greater than appropriate. Likewise, if a 50 ml syringe is disposed in a pump intended for a 10 ml syringe, the applied constant force would be too little and the intended flow would not be provided.

Indeed, U.S. Pat. No. 10,376,636 entitled Compact Mechanical Pump specifically teaches that each embodiment of the Compact Mechanical Pump as disclosed is intended for a syringe of a specific size, and “if the user attempts to use a 30 mL syringe in a pump suited for a 20 ml syringe, the rejection ramps will eject the syringe from the base upon attempted closure of the cover.”

Hence there is a need for a method and system that is capable of overcoming one or more of the above identified challenges.

### SUMMARY OF THE INVENTION

Our invention solves the problems of the prior art by providing novel systems and methods for a mechanical constant force pump assembly as a constant force syringe pump accommodating syringes of different sizes.

In particular, and by way of example only, according to one embodiment of the present invention, provided is a constant force syringe pump assembly accommodating syringes of different sizes comprising: a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base; an adjustable driver structured and arranged to provide a constant force between the pusher and the puller, the adjustable driver provided by a spring connected between the pusher and the puller and providing a first force and an adjustable braker structured and arranged to provide an adjustable braking force to reduce the first force, the constant force adjustably selected by adjusting the adjustable braking force applied to reduce the first force; whereby slidably moving the puller distally when the syringe is seated in the base causes the pusher to contact and exert a substantially constant force on the head of the plunger, the constant force sufficient to move the plunger of the syringe and dispense a liquid from the chamber at a desired flow rate; and a cover connected to the base, a linkage coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

In yet another embodiment, provided is a constant force syringe pump assembly accommodating syringes of different sizes comprising: an expandable base having a proximal end and a distal end, the base comprising: a first base section and a second base section, wherein the first base section is in sliding engagement with the second base section such that the first base section and the second base section are slidably movable relative to each other between a compacted position and an expanded position, wherein the base in the expanded position is adapted to seat a syringe having a plunger slidably disposed within a chamber having an outlet, the chamber having a length and a diameter, the plunger having a head; a pusher in sliding engagement with the base, wherein the pusher is dimensioned to contact the head of the plunger; a puller in sliding engagement with the base; an adjustable driver structured and arranged to provide a constant force between the pusher and the puller, the adjustable driver provided by a spring connected between the pusher and the puller and providing a first force and an adjustable braker structured and arranged to provide an adjustable braking force to reduce the first force, the constant force adjustably selected by adjusting the adjustable braking force applied to reduce the first force; whereby slidably moving the puller distally when the syringe is seated in the base causes the pusher to contact and exert a substantially constant force on the head of the plunger, the constant force sufficient to move the plunger of the syringe and dispense a liquid from the chamber at a desired flow rate; and an expandable cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

Yet for another embodiment, provided is a constant force syringe pump assembly accommodating syringes of different sizes comprising: a base having a proximal end and a distal end, the base structured and arranged to receive a syringe having a plunger slidably disposed within a chamber having an outlet, the chamber having a length and a diameter, the plunger having a head; a pusher in sliding engagement with the base, the pusher structured and arranged to contact the head of the plunger; a puller in sliding engagement with the base; an adjustable driver structured and arranged to provide a constant force between the pusher and the puller, the adjustable driver provided by a spring connected between the pusher and the puller and providing a first force and an adjustable braker structured and arranged to provide an adjustable braking force to reduce the first force, the constant force adjustably selected by adjusting the adjustable braking force applied to reduce the first force; whereby slidably moving the puller distally when the syringe is seated in the base causes the pusher to contact and exert a substantially constant force on the head of the plunger, the constant force sufficient to move the plunger of the syringe and dispense a liquid from the chamber at a desired flow rate; and a cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and tensions the at least one selectively attached spring.

### BRIEF DESCRIPTION OF THE DRAWINGS

FIG. 1 is an upper perspective view of a Constant Force Pump For Different Syringe Sizes in a closed condition in accordance with at least one embodiment of the present invention.

FIG. 2 is an upper perspective view of a Constant Force Pump For Different Syringe Sizes in an open condition in accordance with at least one embodiment of the present invention.

FIGS. 3A-3DD present simplified conceptualizations of an at least one embodiment of a sizer of the Constant Force Pump For Different Syringe Sizes for determining the size of a syringe chamber in accordance with at least one embodiment of the present invention.

FIGS. 4A-4D provides enlarged alternating perspective and elevation views of an adjustable driver of a Constant Force Pump For Different Syringe Sizes shown in FIG. 1 in accordance with at least one embodiment of the present invention.

FIG. 5 is an upper perspective view of a Constant Force Pump For Different Syringe Sizes in an open condition in accordance with another embodiment of the present invention.

FIGS. 6A and 6AA present simplified conceptualizations of yet another embodiment of a sizer of the Constant Force Pump For Different Syringe Sizes for determining the size of a syringe chamber in accordance with at least one embodiment of the present invention

FIGS. 7A and 7B present an enlarged perspective and plain view of the adjustable driver for the embodiment of the Constant Force Pump For Different Syringe Sizes shown in FIG. 5 in accordance with at least one embodiment of the present invention.

FIGS. 7C and 7D present enlarged cut through plain views of the adjustable driver for the embodiment of the Constant Force Pump For Different Syringe Sizes shown in FIG. 5

FIG. 8A is an upper perspective view of a Constant Force Pump For Different Syringe Sizes in an open condition in accordance with yet another embodiment of the present invention.

FIGS. 8B-8E provides enlarged alternating perspective and elevation views of an adjustable driver of a Constant Force Pump For Different Syringe Sizes shown in FIG. 8A in accordance with at least one embodiment of the present invention.

FIG. 9A is an upper perspective view of a Constant Force Pump For Different Syringe Sizes in an open condition in accordance with still yet another embodiment of the present invention.

FIG. 9B is an enlarged perspective views of an adjustable driver of a Constant Force Pump For Different Syringe Sizes shown in FIG. 9A in accordance with at least one embodiment of the present invention.

FIG. 10 is an enlarged perspective views of yet another adjustable driver of a Constant Force Pump For Different Syringe Sizes in accordance with at least one embodiment of the present invention.

FIG. 11 provides a flow diagram of a method of using a Constant Force Pump For Different Syringe Sizes for infusion therapy.

### DETAILED DESCRIPTION

Before proceeding with the detailed description, it is to be appreciated that the present teaching is by way of example only, not by limitation. The concepts herein are not limited to use or application with a specific system or method for a compact mechanical syringe pump for dispensing liquids. Thus, although the instrumentalities described herein are for the convenience of explanation shown and described with respect to exemplary embodiments, it will be understood and appreciated that the principles herein may be applied equally in other types of systems and methods involving a compact mechanical syringe pump. Moreover, the features described or illustrated in connection with one embodiment may be combined with other embodiments, and such combinations, modification and variations are intended to be within the scope of the present invention.

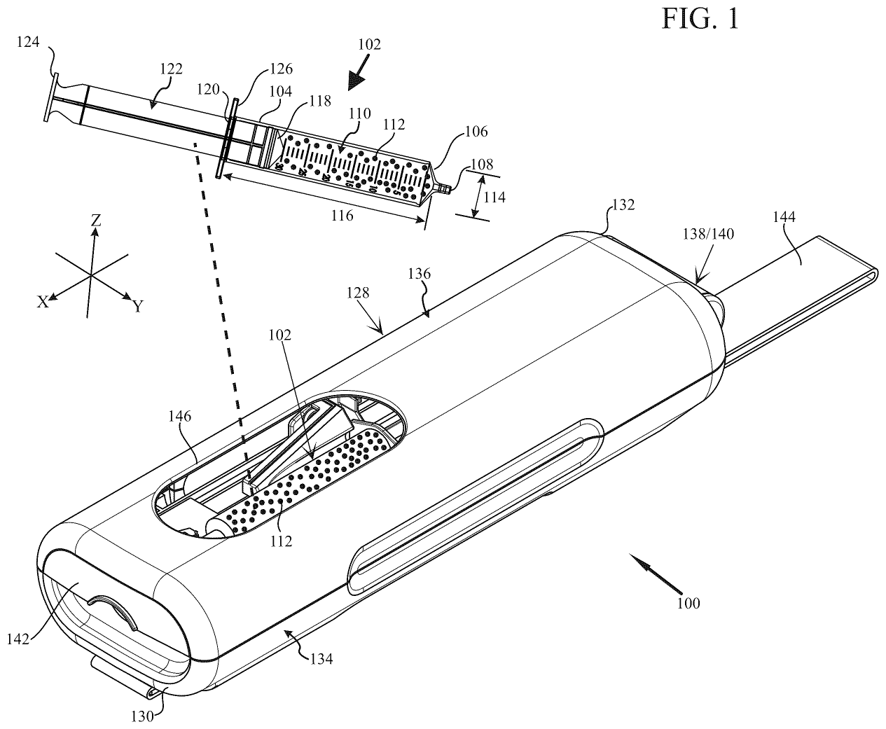

This invention is described with respect to preferred embodiments in the following description with reference to the Figures, in which like numbers represent the same or similar elements. Further, with the respect to the numbering of the same or similar elements, it will be appreciated that the leading values identify the Figure in which the element is first identified and described, e.g., element 100 appears in FIG. 1.

Turning now to the drawings, and more specifically FIG. 1, there is shown a mechanical constant force pump as Constant Force Pump For Different Syringe Sizes, hereinafter CFPDSS 100, for dispensing liquids, e.g., therapeutic liquids, from a variety of different sized syringes in accordance with at least one embodiment of the present invention. As shown, CFPDSS 100 is in a closed position with a syringe 102 disposed inside.

To facilitate the description of systems and methods for embodiments of CFPDSS 100, the orientation of CFPDSS 100 as presented in the figures is referenced to the coordinate system with three axes orthogonal to one another as shown in FIG. 1. The axes intersect mutually at the origin of the coordinate system, which is chosen to be the center of CFPDSS 100, however the axes shown in all figures are offset from their actual locations for clarity and ease of illustration.

As stated, CFPDSS 100 is structured and arranged to advantageously operate with a variety of different sized syringes, of which syringe 102 is merely exemplary. For ease of discussion and illustration, syringe 102 has been illustrated both disposed within the CFPDSS 100, and removed for ease of identification of various elements.

It will be understood and appreciated that, in general, a typical preloaded syringe 102 for use in an infusion treatment may be generally described as having a syringe barrel 104 which at a first end 106 provides an outlet 108 suitable for attachment to, or already fitted with, tubing suitable for administering the liquid in the syringe barrel 104. Normally, this first end 106 of the syringe 102 is tapered so as to reduce the overall size of the syringe 102 from the diameter of the syringe barrel 104 to one suitable for fluid connection with tubing.

The barrel 104 of the syringe 102 also defines the chamber 110 of the syringe 102 which contains the liquid medication (shown as dots 112) to be dispensed. The diameter 114 of the chamber 110 is generally consistent along its length 116, and in most cases the length 116 of the chamber 110 and the diameter 114 of the chamber 110 are substantially the same as the diameter and length of the syringe barrel 104, save for the addition of the thickness of the material from which the syringe barrel 104 is formed. Moreover, unless specifically stated otherwise, it will be understood and appreciated that for CFPDSS 100 as set forth and described herein, the diameter 114 of the barrel 104 and diameter 114 of the chamber 110 may be used interchangeably, likewise the length 116 of the barrel 104 and length 116 of the chamber 110 may be used interchangeably

A plunger seal 118 is disposed in the open end 120 of the syringe barrel 104, opposite from the first end 106. A plunger 122 extends rearward from the plunger seal 118 and is typically about the same length as the syringe barrel 104. As pressure is applied to the head 124 of the plunger 122, the plunger seal 118 is advanced towards the first end 106 of the syringe 102, and the liquid (shown as dots 112) within the syringe 102 is expelled through the outlet 108.

In general, a syringe 102 provides finger tabs 126 disposed proximate to the open end 120 of the syringe 102 such that a user may grasp the tabs with his or fingers and then exert pressure upon the head of the syringe 102 with his or her thumb, palm, or other hand. In other words, the finger tabs 126 provide a brace for the syringe barrel 104 as the plunger seal 118 is driven into and through the syringe barrel 104 by the plunger 122.

As useful as the finger tabs 126 may be, it will be understood and appreciated that the first end 106 of the syringe 102 may also be used as a base during the extrusion process. In some cases, the first end 106 may be fitted with a luer, such as a disc luer, in which case the disc portion of the luer may act as the base during the extrusion process. For at least one embodiment, the luer is a flared luer as set forth in U.S. Pat. No. 10,500,389 entitled, SYSTEM AND METHOD FOR FLARED LUER CONNECTOR FOR MEDICAL TUBING, incorporated herein by reference. For yet another embodiment, the lure is a tapered luer as set forth in U.S. Provisional Application 63/616,368 entitled, SYSTEM AND METHOD FOR A TAPERED LURE CONNECTOR FOR MEDICAL TUBING, incorporated herein by reference.

Moreover, to extrude the liquid from the syringe 102, the syringe barrel 104 must be braced by some element and the plunger 122 must be braced. A force is then applied to move the plunger 122 towards the first end 106. Although in handheld operation, the user may actually move the two components, it will also be understood and appreciated that the first end 106 can be pushed backwards towards the plunger 122, or the plunger 122 can be pushed forwards toward the first end 106.

In simple terms, it is an advantageous aspect of the CFPDSS 100 to receive a syringe 102 as has just been described, brace the first end 106 of the syringe 102 and brace the head 124 of the plunger 122, and then apply a constant force as between the first end 106 and the head 124 of the plunger 122 so as to drive the plunger seal 118 forward and extrude the liquid from the syringe 102. It will also be understood and appreciated, that for at least one alternative embodiment, the syringe 102 is braced by the finger tabs 126 rather than the first end 106. For such an embodiment, as a constant force is applied to the head 124 of the plunger 122, the bracing by the finger tabs 126 ensures that the plunger seal 118 is driven forward towards the first end 106 to extrude the liquid from the syringe 102.

For at least one embodiment, the CFPDSS 100 builds from the Applicant's prior developments with compact mechanical pumps as set forth in U.S. Pat. No. 10,376,636, which is incorporated herein by reference. However, whereas the '636 patent specifically teaches that if a person should try to dispose a 30 ml syringe in a pump suited for a 20 ml Syringe, ejection ramps will eject the Syringe from pump housing when the user attempts to close the housing, the present invention advantageously accommodates syringes 102 of different sizes.

Indeed, embodiments of CFPDSS 100 incorporate many of the features and elements from Applicant's prior developments with compact mechanical pumps set forth in US Patent Application

In addition, CFPDSS 100 incorporates many features and elements from Applicant's prior developments with compact mechanical pumps set forth in U.S. patent application Ser. No. 18/767,796 entitled SYSTEM AND METHOD FOR A CONSTANT FORCE SYRINGE PUMP ACCOMMODATING SYRINGES OF DIFFERENT SIZES, incorporated herein by reference. Whereas the Ser. No. 18/767,796 teaches advantageous systems and methods to automatically select a plurality of different springs to provide an appropriate constant force for a syringe 102, as will be appreciated from the continuing description below, the present invention advantageously applies an appropriate constant force to a syringe by employing an adjustable driver incorporating a spring and an adjustable braker.

Returning to FIG. 1, for at least one embodiment, the CFPDSS 100 has a housing 128 having a distal end 130 and a proximal end 132. For at least one embodiment, the housing 128 is provided by a base 134 and a cover 136 that are hinged at the proximal end 132 to permit opening and closing. For at least one embodiment, the base 134 and a cover 136 are fixed in size. For yet another embodiment, the base 134 and a cover 136 are both expandable.

For at least one embodiment of an expandable CFPDSS 100, the base 134 is provided by a first base section and a second base section, wherein the first base section is in sliding engagement with the second base section such that the first base section and the second base section are slidably movable relative to each other between a compacted position and an expanded position. Likewise, the cover 136 is provided by a first cover section and a second cover section, wherein the first cover section is in sliding engagement with the second cover section such that the first cover section and the second cover section are slidably movable relative to each other between a compacted position and an expanded position. In general, the base 134 and cover 136 transition between compact and expanded states at the same time. Embodiments of such a compacting/expanding base 134 and cover 136 may be extrapolated from '636 patent noted above.

For at least one embodiment, the CFPDSS 100 also includes a dampener 138 to dampen the opening motion of the cover 136. In varying embodiments, the dampener 138 may be separate from the hinge 140 and pivotally coupled to base 134 and a cover 136, or integrated as an element of the hinge 140. Whether a separate element from the hinge 140 or an incorporated part of the hinge 140, for at least one embodiment the dampener 138 is selected from a viscus dampener, a fluid dampener, a rotary friction dampener or the like. For at least one embodiment, the hinge 140 is a unidirectional friction hinge providing a first resistance in a closing operation, and a second resistance in an opening operation, the second resistance greater than the first. More specifically, the hinge 140 may be advantageously structured and arranged to ensure that the cover does not snap up suddenly when the CFPDSS 100 is opened.

Further still, in varying embodiments, the CFPDSS 100 may include a latch or lock assembly 142 such as a safety clasp, for automatically binding the cover 136 in a closed position upon the base 134, and requiring a user to activate a release to open the cover 136.

Further still, for at least one embodiment, CFPDSS 100 also includes a strap 144 to facilitate ease in carrying, hanging, and otherwise using and transporting the CFPDSS 100. Still further, for at least one embodiment CFPDSS 100 provides a window 146 so that a person can observe the state of the syringe 102 disposed within CFPDSS 100.

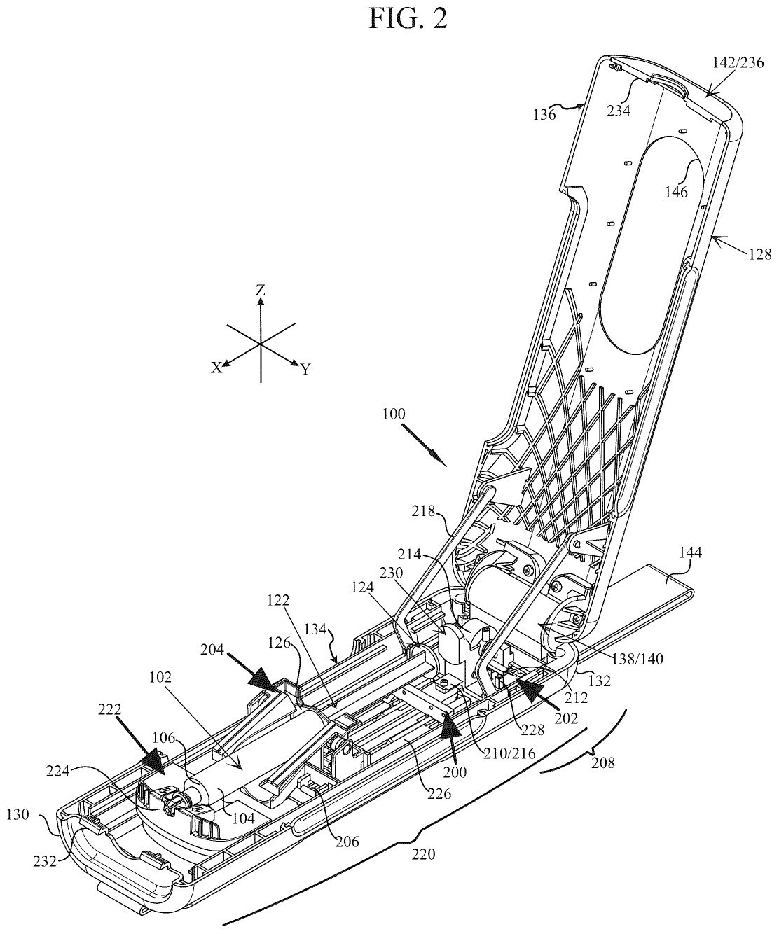

Turning to FIG. 2, an embodiment of CFPDSS 100 is now shown in an open upper perspective view permitting an appreciation of at least some of the elements within CFPDSS 100 which advantageously permit CFPDSS 100 to provide the correct constant force for a plurality of different sized syringes that may be disposed within CFPDSS 100.

As shown, in FIG. 2, within the housing 128, and more specifically disposed upon the base 134 are at least a puller 200, a pusher 202, and a collar 204. It will be appreciated that for at least one embodiment, at least the puller 200, a pusher 202 are slidingly engaged upon at least one track 206.

There is also an adjustable driver 208 structured and arranged to provide a constant force between the pusher 202 and the puller 200. The adjustable driver 208 is provided by at least one spring 210 and at least one adjustable braker 212. For at least one embodiment the spring 210 is a tape spring, disposed within a housing 214 disposed upon the pusher 202, the first end 216 of the spring 210 affixed to the puller 200. It will be understood and appreciated that in varying embodiments, a plurality of springs 210 with one or more adjustable brakers 208 may be used without departing from the scope of the present invention.

It will also be appreciated that the puller 200 has movable linkage 218, linking the puller 200 with the cover 136, such that movement of the cover 136 between an open and closed position imparts sliding motion of the puller 200 along the track(s) 206 of the base 134. More specifically, as the cover 136 is lowered for closing, the linkage 218 moves the puller 200 towards the distal end 130, and when the cover 136 is raised for opening, the linkage 218 moves the puller 200 towards the proximal end 132.

Although for the conceptual embodiment as shown, the cover 136 and base are pivotally connected at one end, an alternative embodiment may be provided where the cover 136 slides longitudinally over the base, the movable linkage 218 linking the puller 200 with the cover 136 being mechanically adapted for activation through the sliding of the cover 136 between an open and closed position as opposed to rotation about a hinge 140.

As the CFPDSS 100 is advantageously structured and arranged to accommodate a plurality of different syringe sizes—e.g., a syringe having a volume of at least 5 milliliters to a syringe having 120 milliliters—it will be understood and appreciated that CFPDSS 100 advantageously permits a different constant force to be applied to each of the different sized syringes. It is this different constant force that is provided by the adjustable driver 208.

In simple terms, it may be understood and appreciated that the spring 210 and the adjustable braker 212 are associated, and cooperatively interact to provide a selected constant force that is appropriate for the syringe 102 that has been disposed within CFPDSS 100. Moreover, the spring 210 provides a first force of attraction between the puller 200 and the pusher 202. For at least one embodiment this first force is the maximum constant force that may be required for a syringe 102 disposed within CFPDSS 100. The adjustable braker 212 provides a braking force that reduces the first force of the spring 210 to the desired constant force required for other syringes requiring less than the maximum constant force.

Moreover, the desired constant force provided by CFPDSS 100, and more specifically the adjustable driver 208, is advantageously between about 1 to 25 pounds of force. As such, the CFPDSS 100 advantageously permits the proper pre-determined constant force appropriate to the size of the syringe chamber to be exerted upon the plunger of the syringe disposed within the enclosure assembly, and for this constant force to be easily changed—increased or decreased—from one syringe to the next when the syringes are of different sizes.

The adjustable braker 212 may be provided in a variety of different mechanical forms, such as but not limited to a friction braker, a staged cam, a pneumatic brake, a progressive brake, a dis brake, or a damper.

In addition, the braking force applied by the adjustable braker 212 applied in at least two ways. In a first case, the braking force may be applied by the adjustable braker 212 directly to the spring 210 thus reducing the first force of the spring to the desired constant force. For at least a second case, the braking force may be applied to the pusher 202 to cause drag against the first force of the spring 210 as the spring 210 acts to pull the pusher 202 towards the puller 200. This drag may be applied by increasing friction between the pusher 202 and the track(s) 206.

In varying embodiments, the braking force applied by the adjustable braker 212 may be manually set or automatically set. For embodiments incorporating systems for automatically setting the adjustable braker, this may be achieved at least in part by a sizer 220—a device structured and arranged to automatically adjust the adjustable braker 212 based on the size of the syringe 102 disposed within CFPDSS 100.

Returning to FIG. 2, and at least a first embodiment of CFPDSS 100, for at least one exemplary embodiment as shown, the collar 204 is disposed in a fixed position upon the base 134, however it will be understood and appreciated that in at least one alternative embodiment, the collar may also be slidingly engaged upon a track or track provided by the base 134.

For the accommodation of different syringes 102 having different barrel diameters, in at least one embodiment the collar 204 may be provided by essentially two side elements that may be adjusted by the user outward or inward to firmly grip the syringe barrel 104. Various alternatives such as, but not limited to, compliant materials that will compress or expand as required by the diameter of the syringe barrel 104 may also be used, to permit the collar 204 to firmly/snuggly grip the syringe barrel and/or provide brace support for the finger tabs of the syringe 102.

For ease of discussion, a syringe 102 is shown as disposed within the CFPDSS 100. More specifically the finger tabs at the open end of the syringe barrel 104 are shown to be engaged with the collar 204, and the first end 106 of the syringe 102 is shown seated with the selector 222.

The selector 222 is selectively movable between multiple positions, which for at least one embodiment are the varying, and distinctly different barrel lengths of a 50 ml syringe 102, a 20 ml syringe 102, a 10 ml syringe 102, and a 5 ml syringe 102.

For at least one embodiment, a spring (not shown) may bias the selector 222 to be disposed proximate to the collar 204. As a selected syringe 102 is disposed in the CFPDSS 100—the first end 106 of the syringe 102 is partially disposed in a seat 224 provided by the selector 220. As the syringe 102 is lowered down to engage the finger tabs 126 with the collar 204, the selector 220 is slid away from the collar 204 to a distinct position appropriate for the size of the syringe 102.

As noted above, for varying embodiments, where the syringe is coupled to a luer, such as the flared luer as set forth in U.S. Pat. No. 10,500,389 entitled, SYSTEM AND METHOD FOR FLARED LUER CONNECTOR FOR MEDICAL TUBING or a tapered luer as set forth in U.S. Provisional Application 63/616,368 entitled, SYSTEM AND METHOD FOR A TAPERED LURE CONNECTOR FOR MEDICAL TUBING, the selector 222, and more specifically the seat 224 of the selector may be structured and arranged as an accepting base for such a tapered or flared luer. Simply put, the seat 224 may be structured and arranged to receive compliant luers and eject non-compliant luers.

For this present example embodiment, the size of the syringe 102 is determined by length 116 of the syringe 102, and more specifically the length 116 of the syringe barrel 104/chamber 110. In such an embodiment, it will be understood and appreciated that the collar 204, which has received the finger tabs 126 and is bracing them is providing the brace for the syringe 102, much as the fingers of a human operator would. For yet another embodiment, the collar 204 and the selector 222 both provide brace for the syringe barrel 104.

It will be understood and appreciated that for at least one embodiment it may be the selector 222 which provides the brace for the syringe 102 in place of the collar 204. Moreover, for at least one embodiment, each distinct position has a binder 222 such that once the selector 222 is slid to a desired position it may be temporarily bind to the base 134 at that position. For at least one embodiment the binder is provided by notches in the base 134 and ridges extending from the selector 222. When the selector 222 is grasped and tilted slightly up, the ridges are removed from the notches and the selector 222 may be moved to a new position. Once repositioned, the selector 222 is pressed down and the ridges are then depressed into corresponding notches such that the selector 222 will remain in the chosen position until it is tilted up once again to disengage the ridges from the notches.

For at least one embodiment, the selector 222 is a component of a sizer 220—which is structured and arranged to determine the size of the chamber of the syringe 102 disposed in the CFPDSS 100, and more specifically seated between the selector 222 and the collar 204. For at least one embodiment, the sizer 220 is a mechanical system provided at least in part by the selector 222 and at least one linkage, gear, control rod or bar that mechanically interconnects the selector 222 with the adjustable driver 208, and more specifically the adjustable braker 212.

For the embodiment shown in FIG. 2, with respect to interconnecting the selector 222 with the adjustable braker 212, the selected position of the selector 222 mechanically triggers one or more controllers 226 to engage one or more braker pins 228 of the adjustable braker 212. It will be understood and appreciated that the one or more controllers 226 may be further described as a control rod/gear/bar/cable or other element capable of interconnecting the selector 222 and the adjustable braker 212.

Indeed, each of the one or more controllers 226, such as but not limited to control rod(s)/bar(s), may be further described as a lever-having a low section and a high section. As the selector 222 is slid longitudinally within the housing 128 along one or more tracks 206, the one or more control rod(s)/bar(s)/lever(s) 226 may be slid a sufficient distance such that the low section transitions to a high section, the high section activating the selection of an associated braker pins 228.

Indeed, it will be understood and appreciated that mechanical determination of the size of the syringe 102 is, for at least one embodiment determined by the movement of the selector 222 towards the distal end 130 as the syringe 102 is disposed into the CFPDSS 100. More specifically, the user disposes the first end 106 of the syringe 102 into the seat 224 of the selector 222 and pushes the selector 222 towards the distal end 130 to bring the finger tabs 126 into position to be disposed into the collar 204. The longitudinal movement of the selector 222 within the housing 128 along the base 134 triggers one or more control rod(s)/bar(s)/lever(s) to selectively engage one or more braker pins 228, which provide a drag force to the pusher 202.

When the cover 136 is closed, the linkage 218 coupled between the cover 136 and the puller 200 operates to drive the puller 200 down the track(s) 212 of the base 134 towards the collar 204 and the distal end 130. As this sliding movement occurs, each engaged braker pin 228 providing a drag force upon the pusher 202 as the spring 210 pulls the pusher 202 towards the puller 200, this drag force reducing the effective first force of the spring to achieve the proper constant force for the size of the syringe that has been disposed within CFPDSS 100.

As the puller 200 will be held substantially in place by the linkage 218 when the cover 136 is closed, it is the pusher 202 which will then slide towards the puller 200. As may be appreciated in FIG. 2 the pusher 202 has a plunger receiver 230 which is structured and arranged to contact the head 124 of the plunger 122. For at least one embodiment, the plunger receiver 230 does not just contact, but engages with the head 124 of the plunger 122 such as by providing a seat or basin which may help to center the plunger 122 and avoid possible slide slippage when and as force is applied to the plunger 122.

The lock assembly 142 may also be more fully appreciated in FIG. 2. More specifically, for at least one embodiment, the lock assembly 142 is provided by engaging teeth 232 provided at the distal end 130 and corresponding teeth 234 on the cover, with plate 236 being biased inward by a spring (not shown), a user using his or her finger to pull the plate outward to disengage the teeth 232 and 234 and allow the cover 136 to open. As noted above, the hinge 140 may be unidirectional friction hinge which advantageously slows the opening of the cover 136

The first force of the spring 210 offset by the braking force provided by the engaged braker pins 228 results in a pre-determined constant force drawing the pusher 202 towards the puller 200, this pre-determined constant force specifically applied to the plunger 122—driving it into the syringe barrel 104 and extruding the liquid from the outlet 108 at the first end 106.

Moreover, the sizer 220—provided by the exemplary embodiment of the selector 222, the control rod(s)/bar(s)/lever(s) 226 and the selected engagement of the first ends 216 of one or more tape springs 210 advantageously ensures that the force provided by the engaged tape springs 210 is a pre-determined forced appropriate for the size of the syringe 102 that has been disposed within CFPDSS 100.

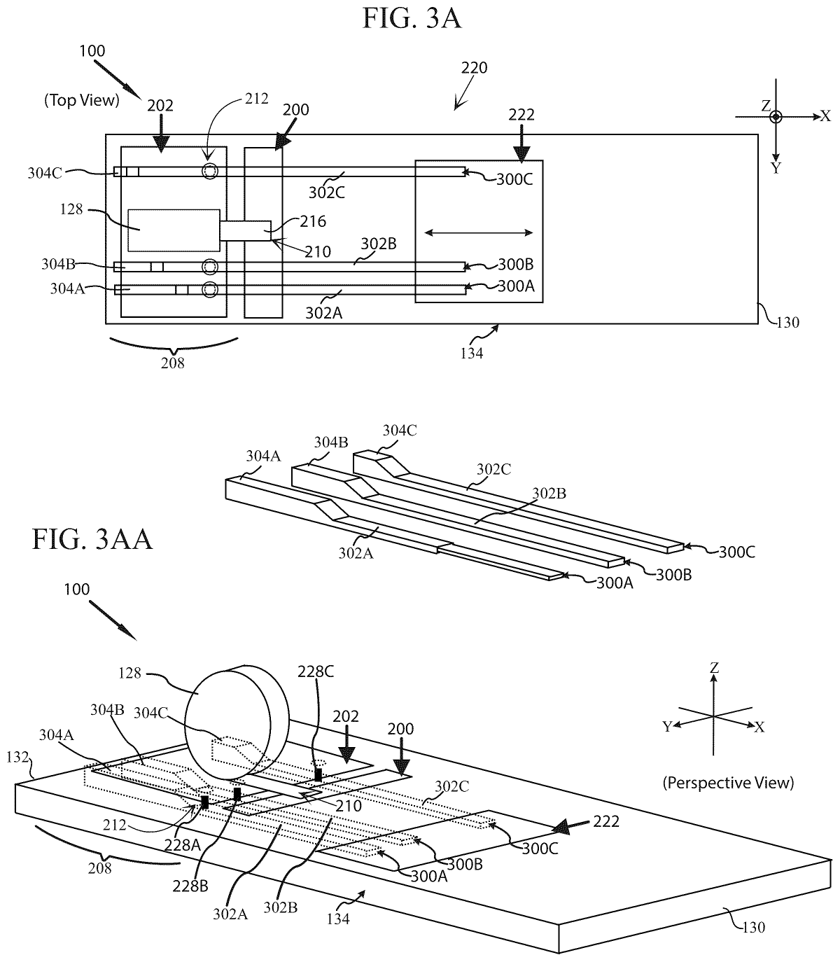

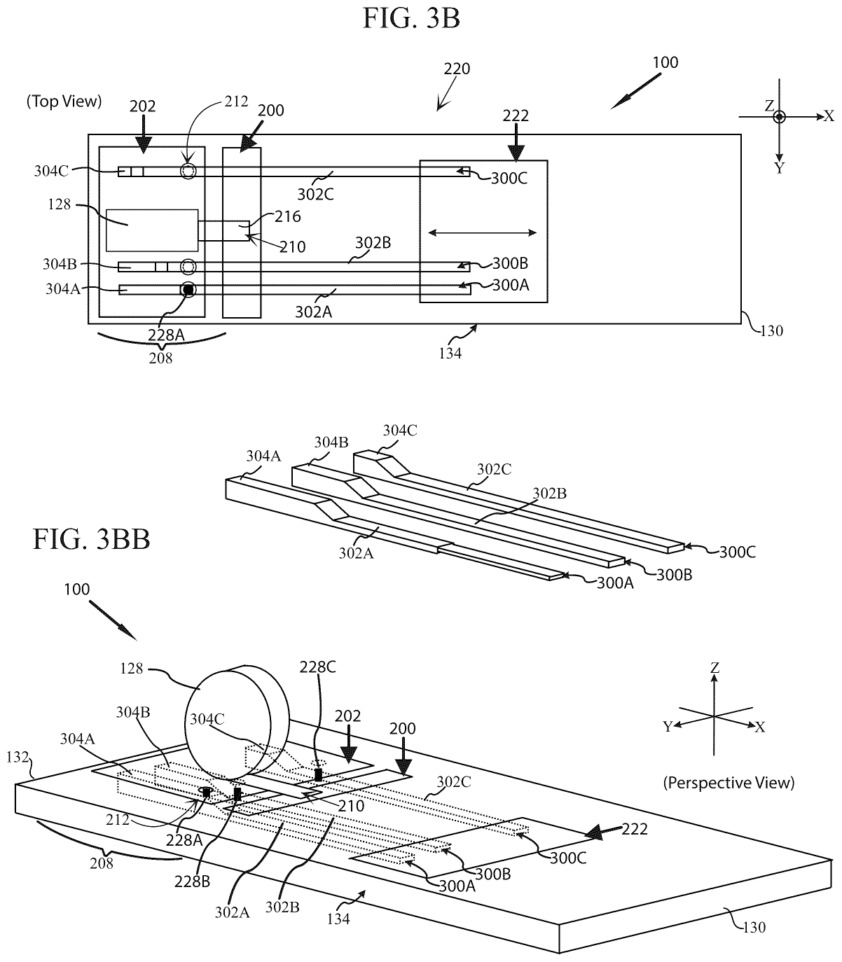

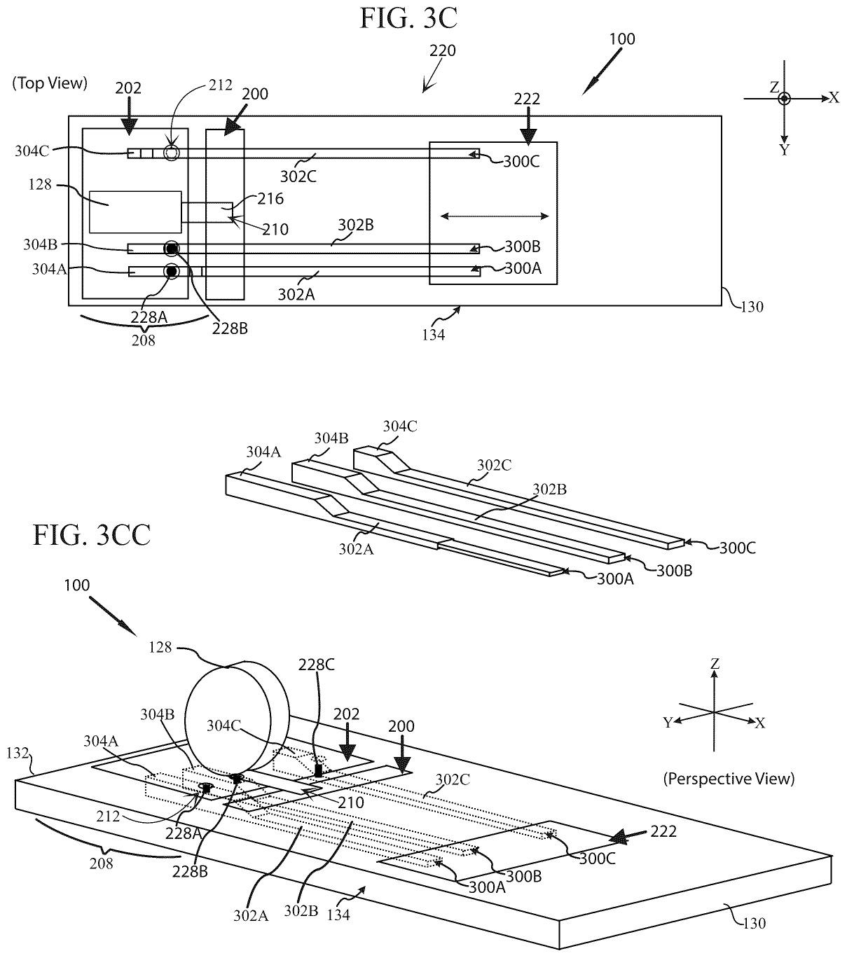

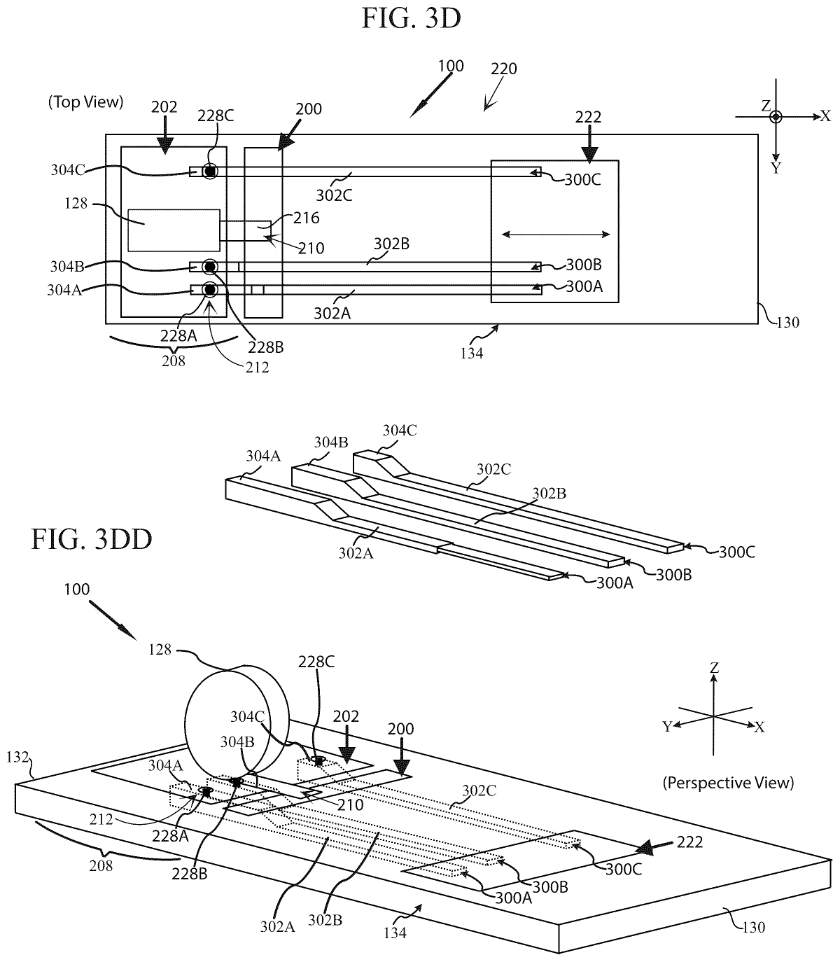

This mechanical selection of braker pins 228 as elements of the adjustable braker 212 may be more fully appreciated with respect to the conceptual illustrations presented in FIGS. 3A-3CC. For ease of illustration and discussion the CFPDSS 100 has been vastly simplified for demonstration purposes. The spring 210 is disposed upon the pusher 202, with a first end of the spring 210 joined to the puller 200. Three control rods/levers 300 corresponding to control rod/lever 226 are also shown.

In each FIG. 3A-3DD, at the top of the drawing page is shown a top view (FIGS. 3A, 3B, 3C and 3D) of the simplified CFPDSS 100 and at the bottom is shown a corresponding perspective view (FIGS. 3AA, 3BB, 3CC and 3DD) of the simplified CFPDSS 100. In the middle of this top and perspective view, a reference view of the control rods 226, which for the present illustrations are identified as control rods 300A, 300B, and 300C.

In each reference view, each control rod 300, and more specifically control rods 300A, 300B, and 300C is appreciated to have a low section 302A, 302B, and 302C and a high section 304A, 304B, and 304C. As may be appreciated from the reference illustrations, each control rod 300 has a different profile as each has a high section 304 of a different length.

In addition, control rod 300A has been illustrated with a telescoping slide arrangement such that a portion of the low section 302A may be slid into the portion of control rod 300A providing the high section 304A. Of course, it will be understood and appreciated that this arrangement may be reversed in an alternative embodiment wherein the portion of the control rod 300A providing the high section 304A slides into the portion of the control rod 300A providing the low section.

For either arrangement, it will be understood and appreciated that these alternative embodiments of control rods 300 would be incorporated in alternative embodiments noted above where the base 134 and cover 136 are also configured to have a sliding arrangement as between a compact for storage and extended for use state of configuration.

In FIGS. 3A & 3AA the system is at rest and no braker pins 228 have been engaged. FIGS. 3B & 3BB, 3C & 3CC, and 3D & 3DD each illustrate how different syringes having different length advantageously mechanically trigger different engagements of braker pins 228.

In FIG. 3B-3BB, conceptualizing the insertion of a syringe 102 having a first length, the selector 222 has advanced longitudinally away from the spring 210 towards the distal end 130 of the base 134 and thus moved control rods/levers 300 towards the distal end 130 as well. As such, control rod/lever 300A has moved such that it has transitioned to a high section 304A below braker pin 228A which is now engaged with pusher 202.

In FIG. 3C-3CC, conceptualizing the insertion of a syringe 102 having a second length, the selector 222 has advanced further towards the distal end 130 of the base 134 and thus moved control rods/levers 300 towards the distal end 130 as well. As such, in addition to control rod/lever 300A having engaged braker pin 228A, control rod/lever 300B has also moved such that it has transitioned to a high section 304B below braker pin 228B which is now engaged with pusher 202.

Continuing with the example, FIG. 3D-3DD, conceptualizes the insertion of a syringe 102 having a third length, the selector 222 has advanced even further towards the distal end 130 of the base 134 and thus moved control rods/levers 300 further towards the distal end 130 as well. As such, control rod/lever 300C has now also moved longitudinally to such a degree that it has transitioned to a high section 304C below braker pin 228C which is also now engaged with pusher 202, in addition to braker pin 228A and braker pin 228B.

For ease of illustration, the conceptual illustrations have been rendered to show the engagement of braker pins 228, however it will be understood and appreciated that the initial state could be reversed, such that at rest all braker pins 228 are initially engaged, and with successively larger/longer syringes 102, the braker pins 228 are selectively disengaged.

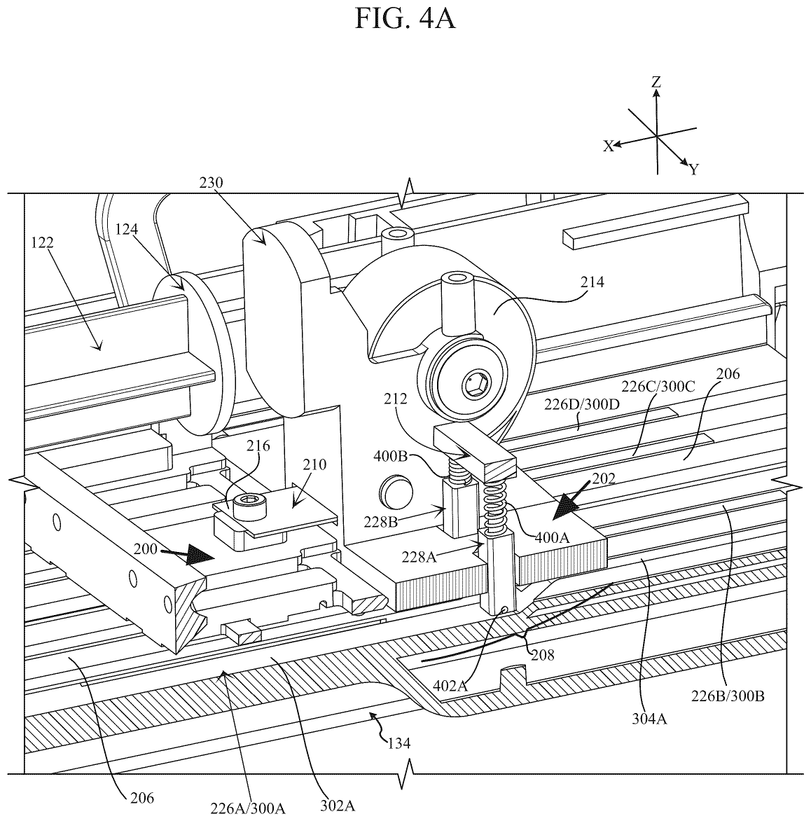

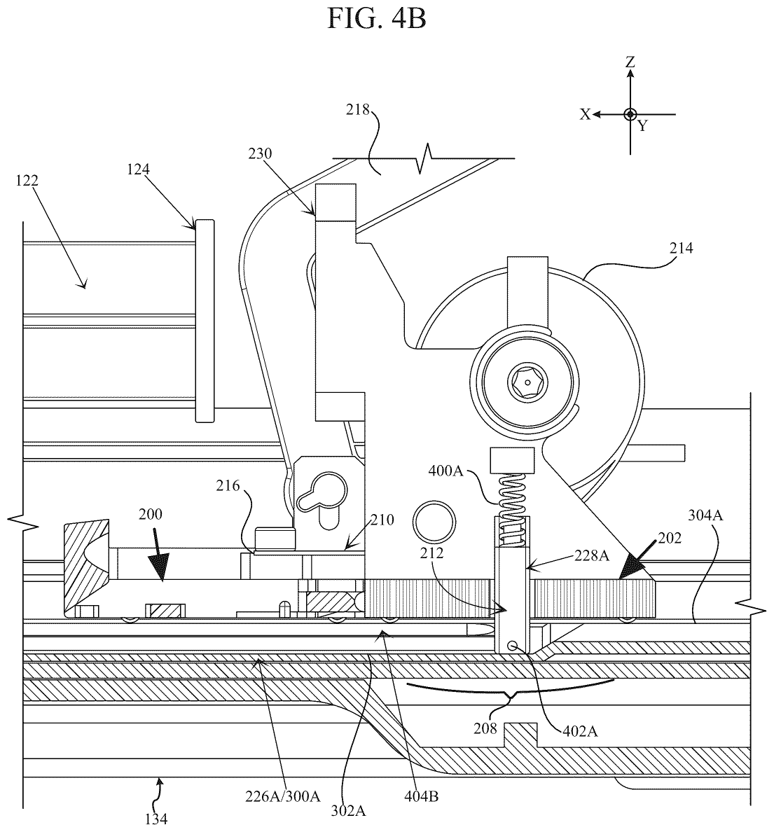

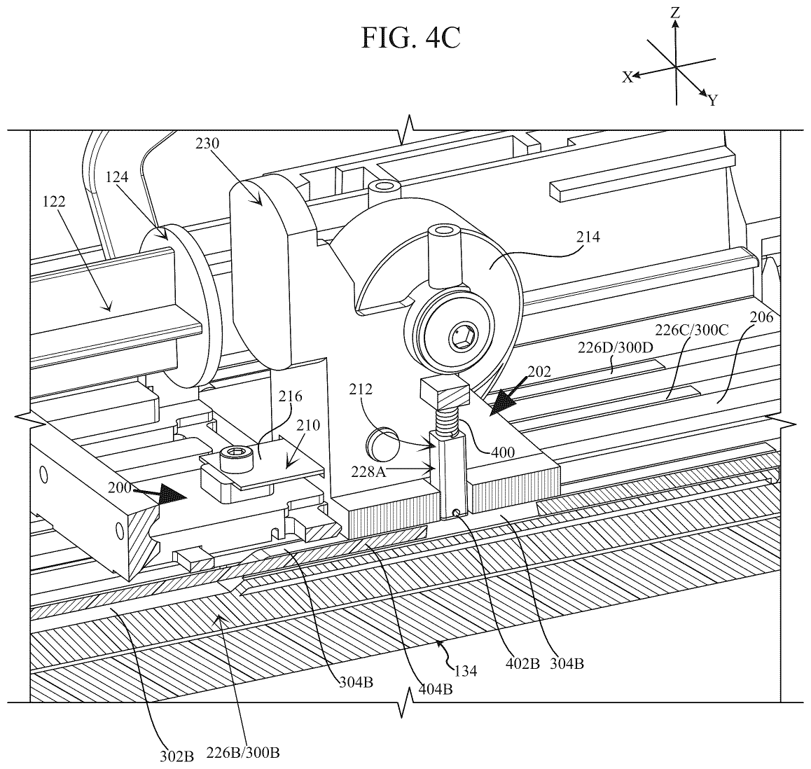

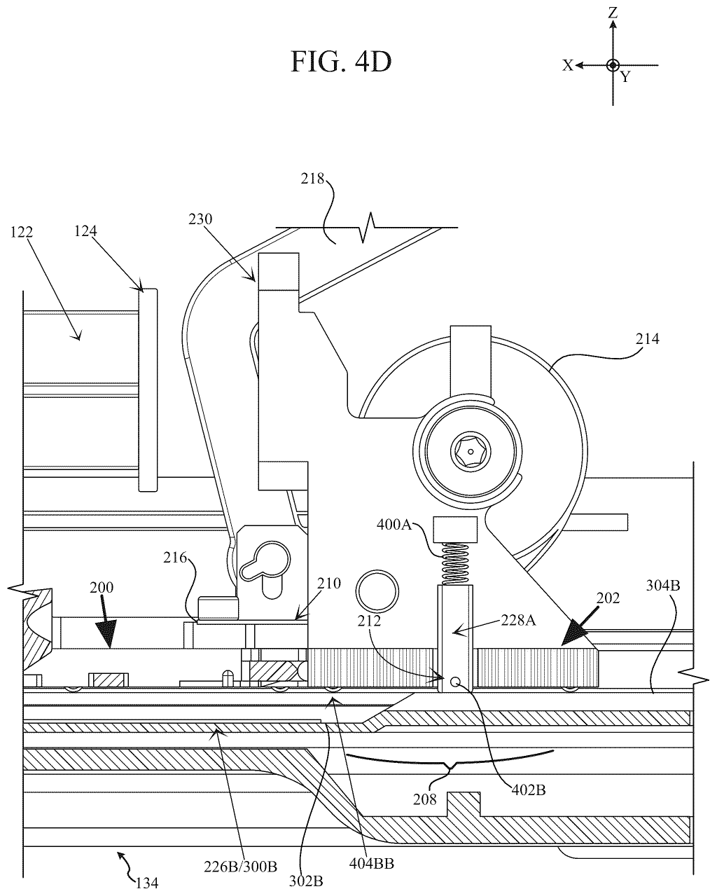

FIGS. 4A-4D present enlarged partial perspective and side illustrations of the control rods/levers 226 and braker pins 228 when at rest and engaged. The first end of the spring 210 may also be appreciated as being permanently affixed to the puller 200, with the housing 214 of the spring 210 affixed to the pusher 202. As the spring 210 is directly coupled between both the pusher 202 and the puller 200, it will be understood and appreciated that the first force of the spring will be applied as a retractive force at such time as the puller 202 and puller 200 are disposed apart from one another. Adjustment to this first force is advantageously achieved by a braking force as may be adjustably applied by the adjustable braker 212. In FIGS. 4A-4D the incorporation of the adjustable braker 212 and more specifically the braker pins 228 as elements of the pusher 202 may be more fully appreciated.

More specifically, in FIG. 4A, presenting a perspective view and FIG. 4B presenting a side view, the CFPDSS 100 has not received a syringe 102 with a length sufficient to trigger control rod/lever 226A/300A for a transition from the low section 302A under braker pin 228A, to a high section 304A which would elevate braker pin 228A, spring 400A expanded. However, braker pin 228B has been elevated, indicating that the length of the received syringe is sufficient to trigger its control rod/lever (not shown in FIGS. 4A and 4B-See FIGS. 4C and 4D), and associated spring 400B now compressed.

In FIGS. 4C, presenting a perspective view and FIG. 4D presenting a side view, control rod/lever 226B is now shown and it may be appreciated that high section 304A is disposed beneath braker pin 228B, such that braker pin 228A has moved vertically upward compressing spring 400A. For at least one embodiment, a rider pin 402B is disposed through the distal end of braker pin 228 which with braker pin 228A now elevated, will ride upon a braker rails 404B & 404BB, when the pusher advances forward when the cover is closed and the linkage 218 (see FIGS. 4B & 4D) advances the puller towards the distal end 130 (not shown in FIGS. 4A-4D). As spring 400A is attempting to expand, it provides a force through braker pin 228B, the friction between the rider pin 402B and the braker rail 404B providing the adjustable braking force providing drag against the first force of spring 210 so as to achieve the desired constant force appropriate for the received syringe 102.

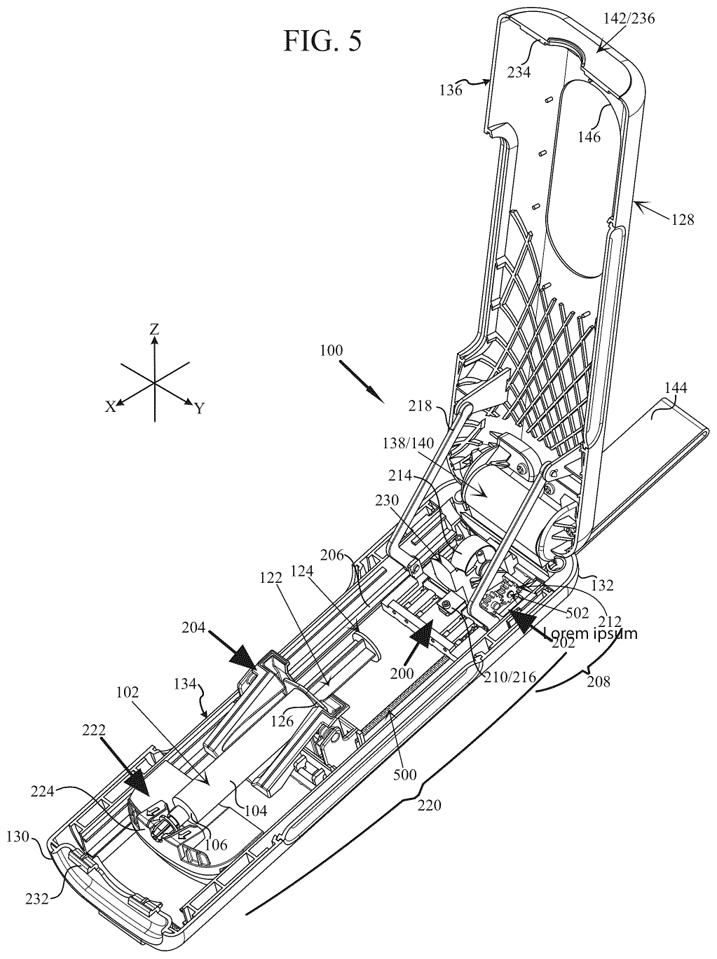

FIGS. 2-4D have conceptually presented at least one embodiment wherein the adjustable braker 212 is structured and arranged to provide a braking force indirectly to the spring 210. FIGS. 5-8E conceptually present at least one alternative embodiment wherein the adjustable braker 212 is structured and arranged to provide a braking force directly to the spring 210.

More specifically, FIG. 5 presents a top perspective view of an open alternative embodiment of CFPDSS 100. As shown, this embodiment of CFPDSS 100 shares essentially the same core elements as described above with respect to the CFPDSS 100 shown in FIG. 2, with the notable exception of having a different adjustable braker 212.

For the alternative embodiment shown in FIG. 5, the selector 222 is coupled to a adjustable braker 212 by a controller that may be more clearly appreciated as a gear rail 500. As the selector 222 is moved towards and away from the distal end 130 the gear rail 500 moves longitudinally beneath the puller 200 and the pusher 202. As shown, the gear rail 500 meshes with adjustment gears 502 which are disposed through the sidewall of the housing 214 of the spring 210. More specifically, longitudinal motion of the gear rail 500 translates to rotational motion of the adjustment gears 502 which in turn cause the motion of an internal cam (not shown) to apply friction to the spring 210 or release friction from the spring 210.

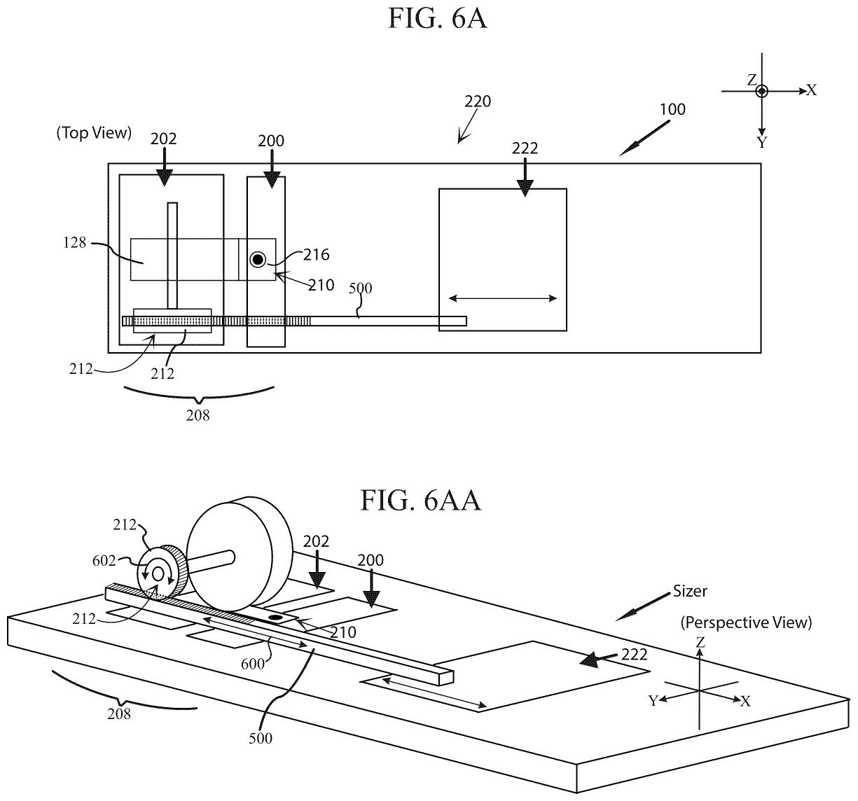

The concept for such an adjustable braker is conceptually shown in FIG. 6A and FIG. 6AA. As with FIGS. 3A-3DD CFPDSS 100 has been vastly simplified for demonstration purposes. FIG. 3A provides a top view and FIG. 3AA provides a corresponding perspective view. The spring 210 is has been shown disposed within housing 214 which is affixed to the pusher 202. The first end 216 of the spring is affixed to the puller 200.

As selector 222 traverses longitudinally towards or away from the distal end 130, the gear rail 500 passes beneath adjustment gear 502, and the longitudinal motion 600 of gear rail 500 translates to rotational motion 602 of adjustment gear 502. For at least one embodiment, this rotational motion 602 is used to rotate a cam either down and against the spring 210 within the housing 214 (providing an increase in the applied adjustable braking force), or it may be used to up and away from the spring 210 within the housing 214 (providing a decrease in the applied adjustable braking force.

For yet another embodiment, the adjustment gear may in turn drive a disc break (not shown) against the spring to increase the adjustable braking force, or away from the spring to reduce the adjustable braking force.

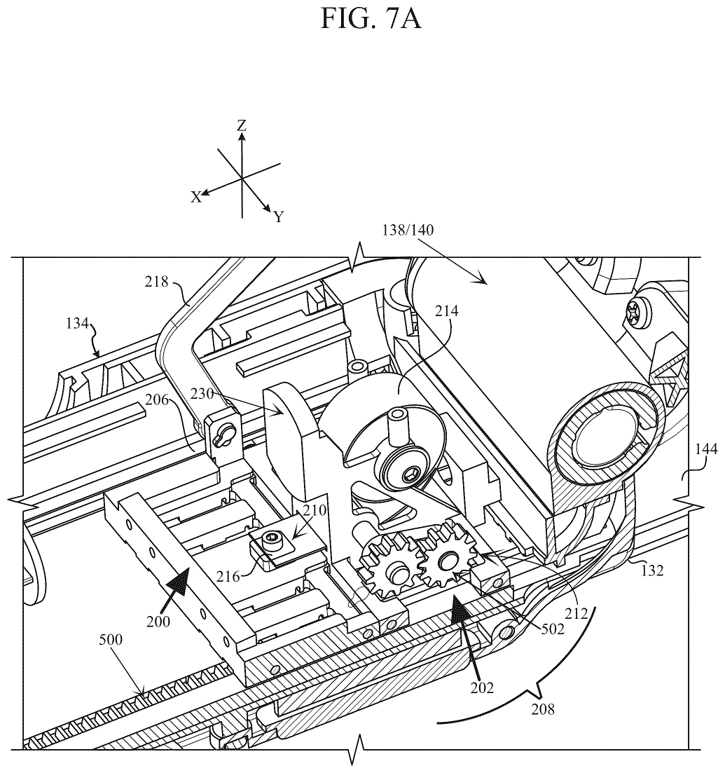

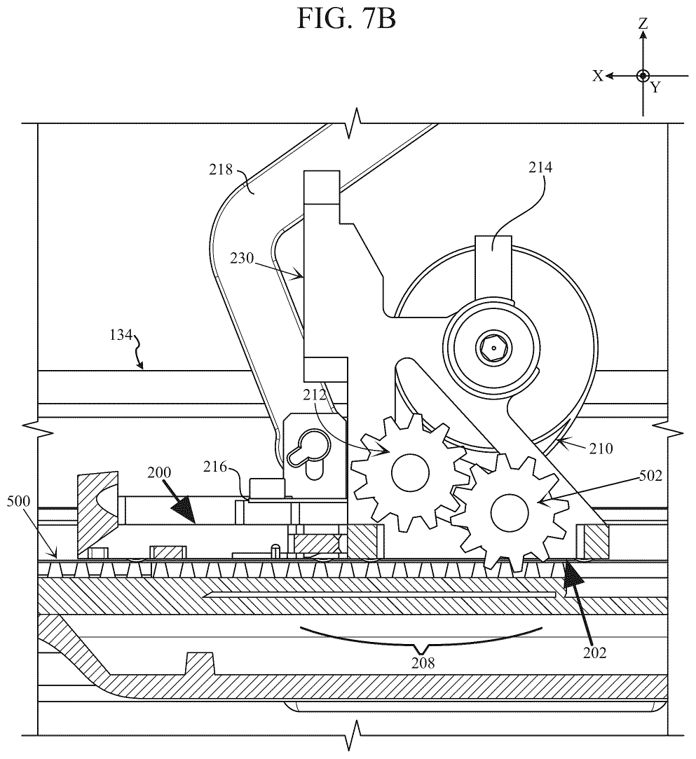

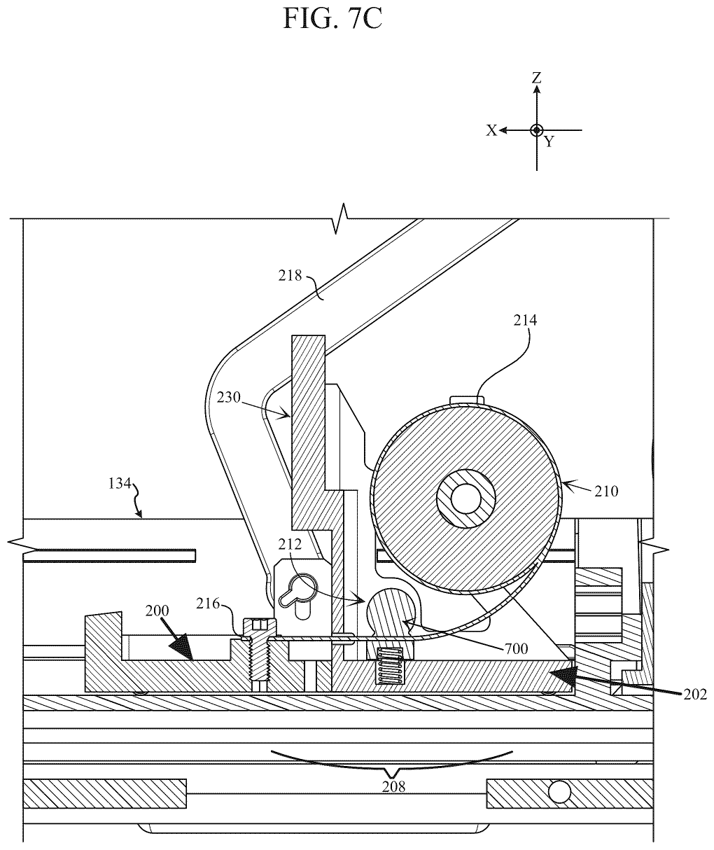

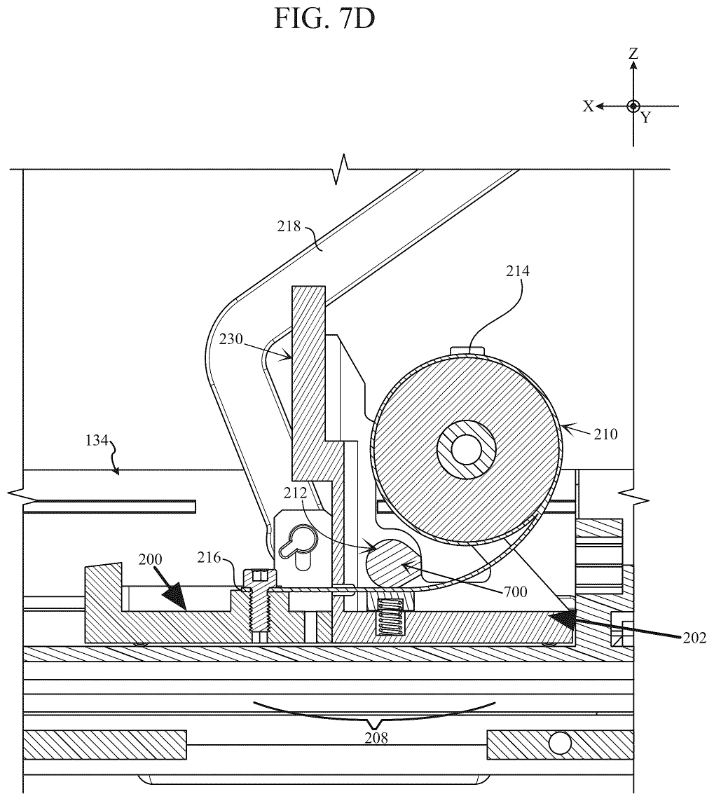

FIG. 7A provides an enlarged partial perspective view of adjustable driver 214, and more specifically the gear rail 500 passing beneath and engaging the adjustment gears 502, with FIG. 7B providing a plain side view as well.

FIGS. 7C and 7D provides cut through views of the adjustable driver 208, further showing an adjustable cam 700 that is driven by the adjustment gears 502. In FIG. 7C it will be appreciated that the cam 700 has been rotated down to engage with the spring 210, and as such is applying an adjustable braking force to the spring 210. In FIG. 7D it will be appreciated that the cam 700 has been rotated substantially off of the spring 210 and as such has been adjusted to apply less of a braking force to the spring 210.

Moreover, it will be understood and appreciated that by varying the degree of contact between the cam 700 and the spring 210 an adjustable amount of braking force is applied directly to the spring 210 so as to achieve the desired substantially constant force appropriate for the syringe 102 disposed within CFPDSS 100.

With respect to FIGS. 2-7D as discussed above, it will be understood and appreciated that for these depicted embodiments, the CFPDSS 100 has automatically determined and adjusted the braking force of the adjustable driver 208 by determining the size of the syringe.

It will be understood and appreciated that the advantages of CFPDSS 100 to accommodate a variety of different sized syringes requiring substantially different constant force for extrusion is not strictly limited to self-determining devices.

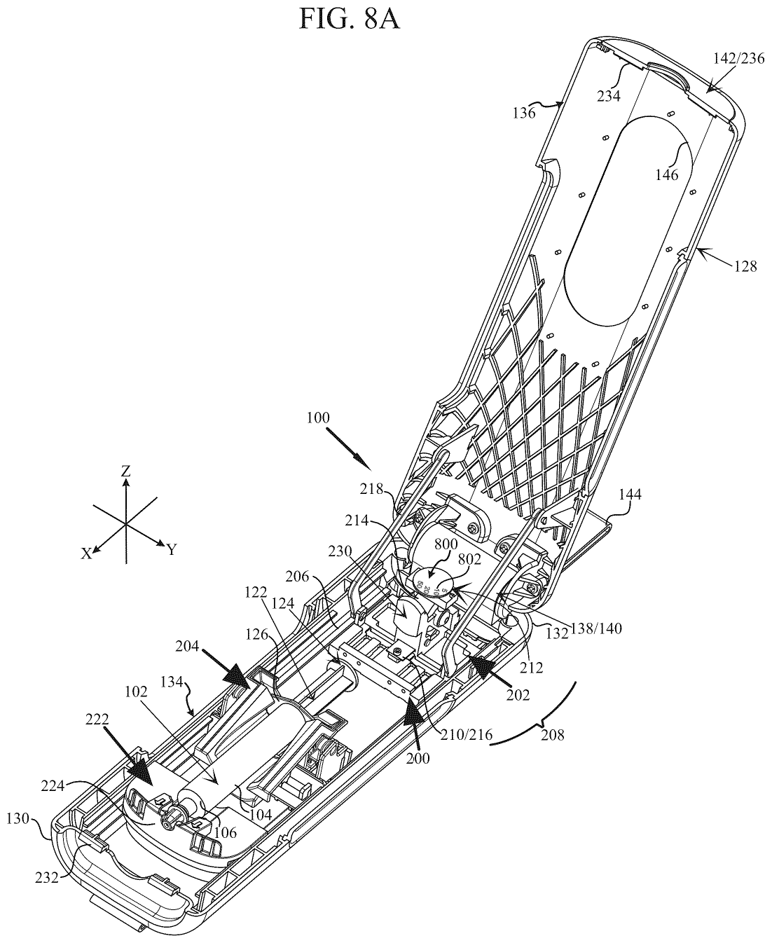

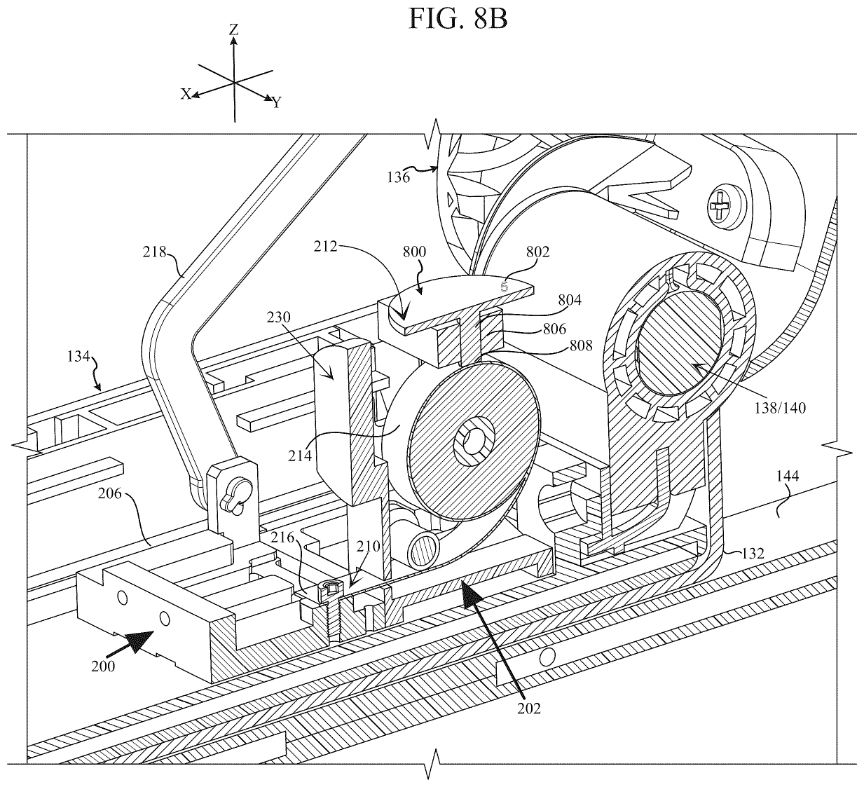

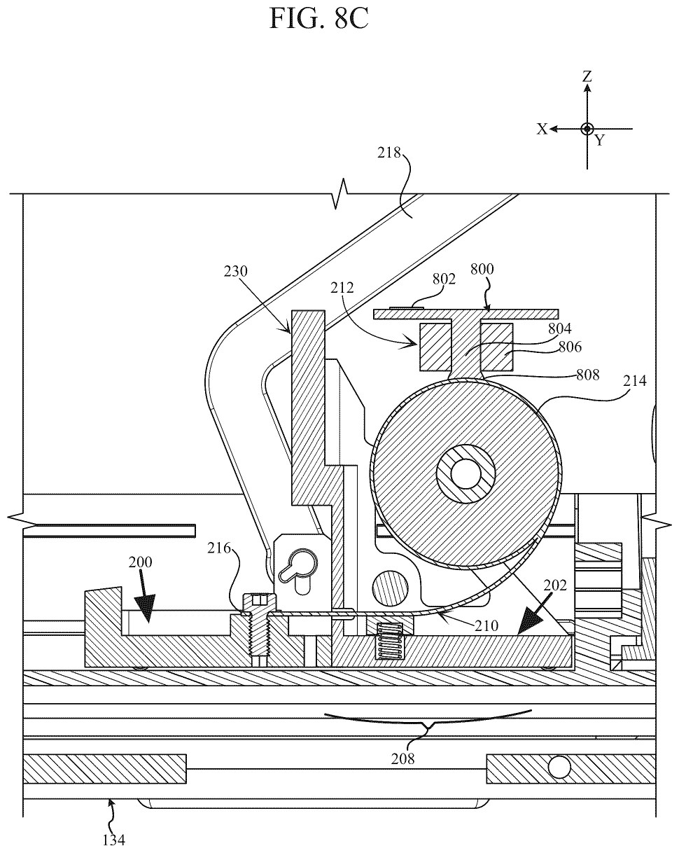

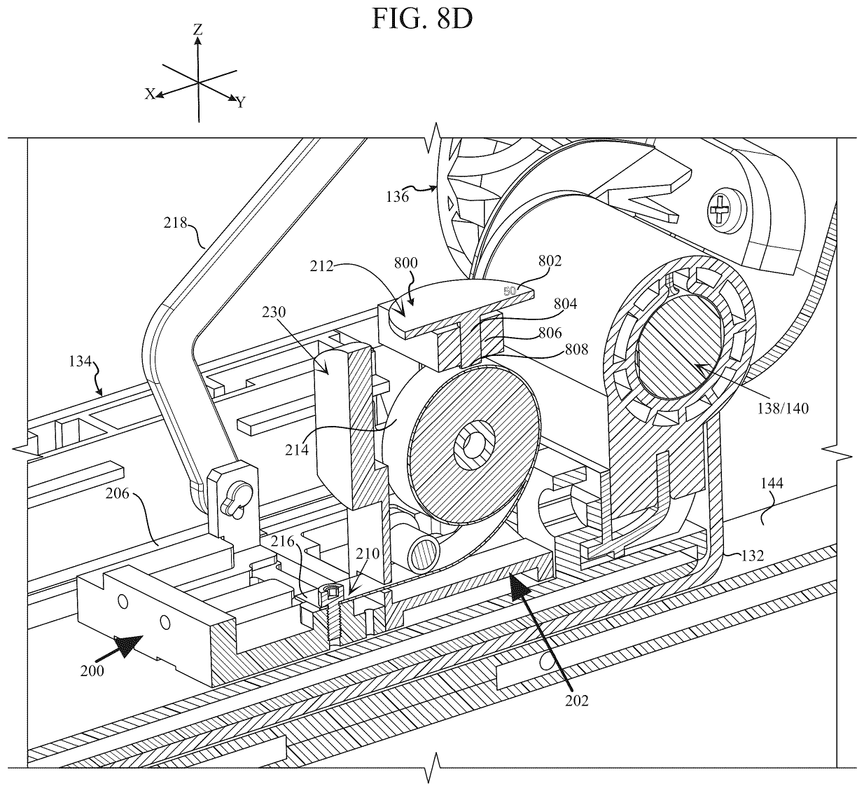

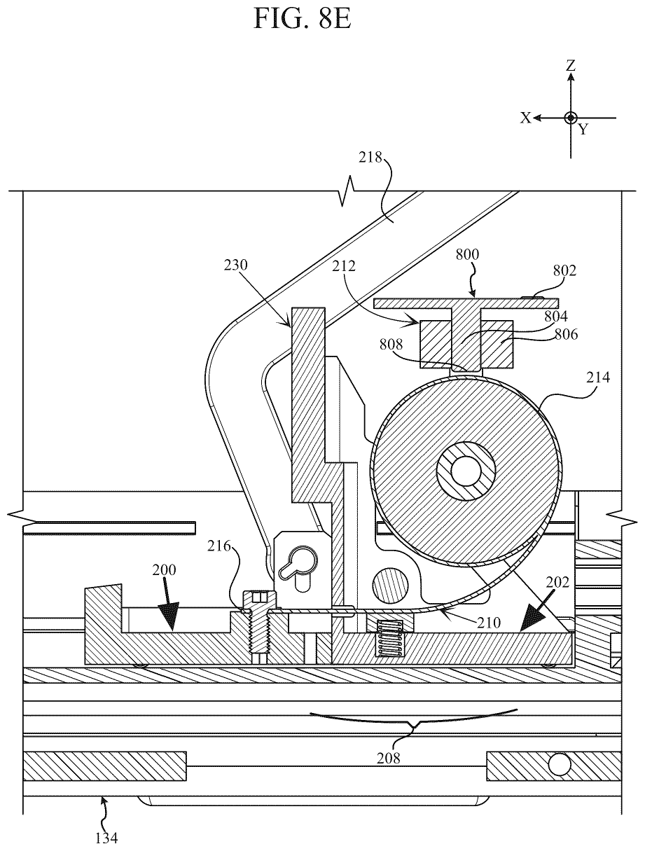

Indeed FIGS. 8A-8E present yet another alternative embodiment where the adjustable driver comprises an adjustable driver that is manually adjusted. More specifically, FIG. 8A presents a top perspective view of an open alternative embodiment of CFPDSS 100. As shown, this embodiment of CFPDSS 100 shares essentially the same core elements as described above with respect to the CFPDSS 100 devices shown and described above with respect to FIGS. 2 and 5, with the notable exception of having a different adjustable braker 212.

In this case it is a manually operated adjustable braker 212. As shown the adjustable braker 212 provides a knob 800 with indicia 802. For at least one embodiment the indicia 802 is visual indicia. For at least one alternative embodiment the indicia 802 may be tactile indicia.

For at least one embodiment, the visual indicia 802 is provided to indicate the size of a syringe, e.g., 50 ml syringe, a 20 ml syringe, a 10 ml syringe, or a 5 ml syringe. For at least one other embodiment the visual indicia 802 indicates a degree or percentage of applied braking force. Of course it will be understood and appreciated that the visual indicia 802 may be a mapping-“A” for syringe “A”, “B” for syringe “B”, “C” for syringe “C”, etc., each syringe identified by a number, letter, character or shape that may be more easily appreciated by some users than an indication of milliliters. Moreover, for at least one embodiment the adjustable braking force is selected by selecting a predefined setting for the adjustable braker 212.

For yet another embodiment, the adjustable braker 212 may be entirely variable. For such embodiments, CFPDSS 100 may be provided with a flow rate indicator, or coupled to a flow rate indicator, the braking force selected by adjusting the adjustable braker 212 to a desired flow rate shown by the flow rate indicator

FIG. 8B is an enlarged cross section view through the adjustable braker 212 shown in FIG. 8A, with FIG. 8C providing an enlarged corresponding plain cut through view. In both FIGS. 8B and 8C it may be appreciated that the knob 800 has a threaded shaft 804 passing through a threaded mount 806. In FIGS. 8B and 8C the knob 800 is in an initial position such that threaded shaft 804 has not rotated sufficiently to bring the distal end 808 of the threaded shaft 804 into contact with the spring 210.

FIG. 8D is an enlarged cross section view through the adjustable braker 212 shown in FIG. 8, with FIG. 8E providing an enlarged corresponding plain cut through view. In these views it will be understood and appreciated that the knob 800 has been rotated to drive threaded shaft 804 through mount 806, and in so doing, the distal end 808 of the threaded shaft 804 is now in contact with the spring 210 and applying an adjustable braking force directly to the spring 210.

For at least one embodiment the distal end 808 of the threaded shaft 804 is a semi-compliant/semi-resilient material, such that it will deform against the spring 210 to increase the surface area of contact between the distal end 808 and the spring 210, thus further permitting adjustment of the adjustable braking force applied.

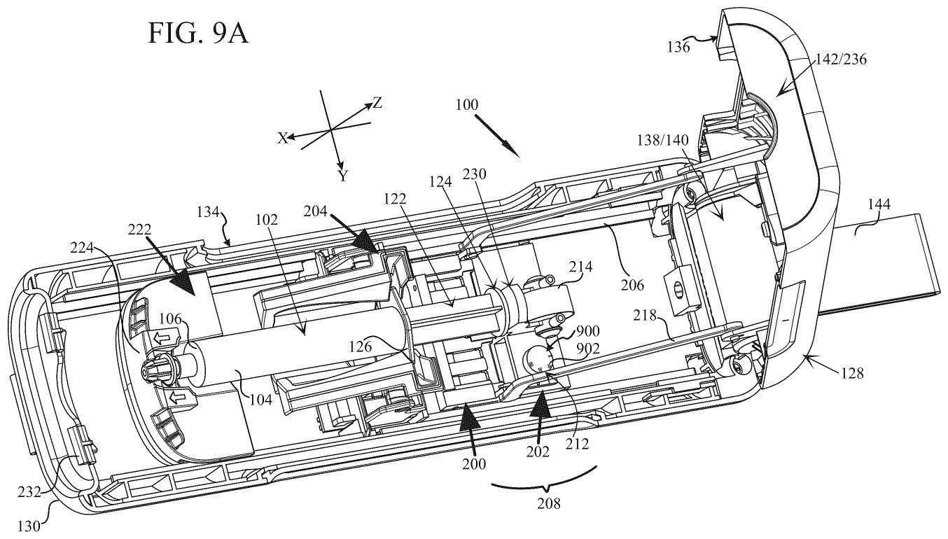

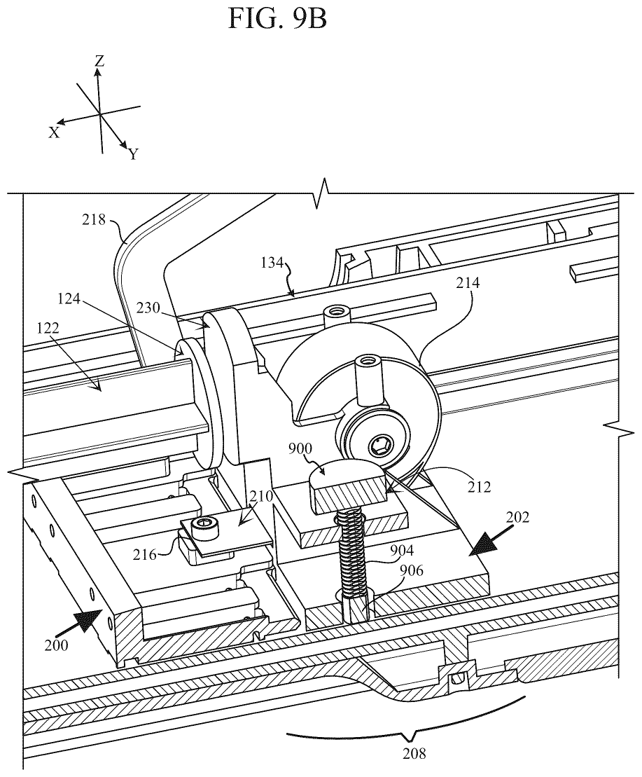

FIG. 9A shows yet another embodiment of CFPDSS 100 with a manually operated adjustable braker 212. FIG. 9B shows an enlarged perspective cross section of the embodiment shown. In FIG. 9A, as with the embodiment shown in FIGS. 8A-8D, here again the adjustable braker is a knob 900 with indicia 902 on a threaded shaft 904, but as with the embodiment of FIG. 1A, the adjustable braking force is applied to the pusher 202, to create frictional drag between the pusher 202 and the rails or base 134 upon which the pusher 202 slides.

As may be more fully appreciated in FIG. 9B, for at least one embodiment, the distal end 906 of the threaded shaft 904 is a semi-compliant/semi-resilient material, such that it will deform against the rail or base 134 to increase the surface area of contact between the distal end 906 and the rail or base 134, thus further permitting adjustment of the adjustable braking force applied.

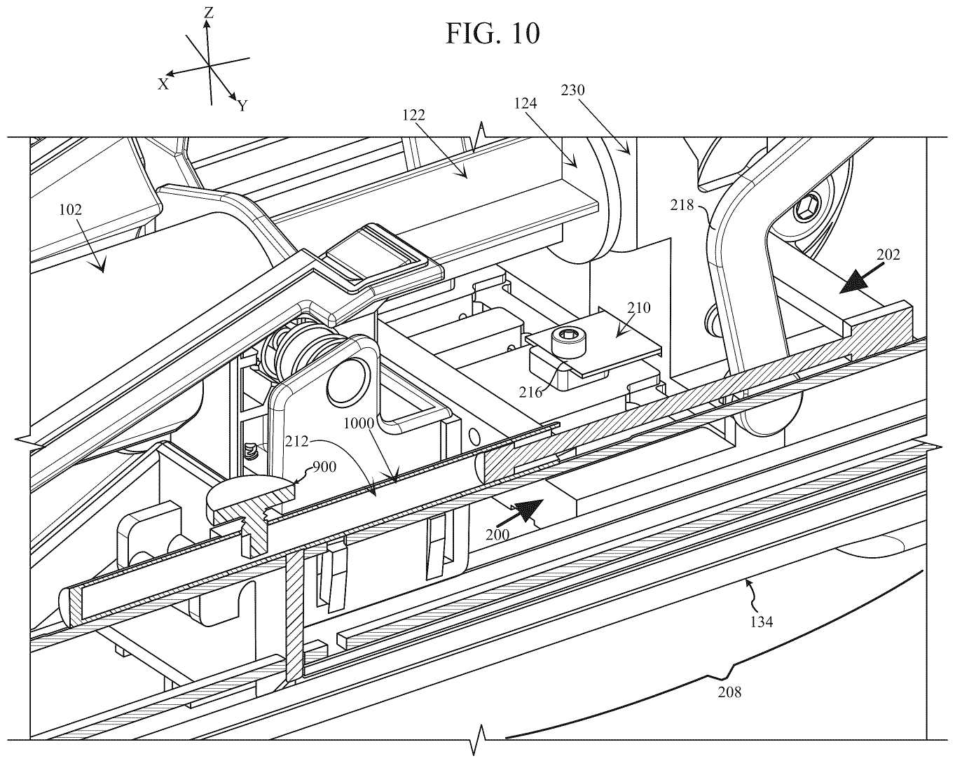

FIG. 10 presents yet another embodiment of CFPDSS 100. For this embodiment, the adjustable driver 208 includes an adjustable braker 212 that is a pneumatic piston 1000, with the inflow or outflow of air governed by an adjustable valve operated by knob 1002.

With respect to the embodiments shown in FIGS. 8A-10 shown as manually operated adjustable brakers, it will be understood and appreciated that variations of each embodiment may be automated by adapting use of the sizer 220 using a selector 222 and variations of control linkage/rods/levers 226 as shown and described above.

With respect to the above description, it may be appreciated that at least one embodiment of a CFPDSS 100 accommodating syringes 102 of different sizes may be summarized as: a base 134 having a proximal end 132 and a distal end 132, the base 134 structured and arranged to receive a syringe 102 having a plunger 122 slidably disposed within a chamber 110 having an outlet 108, the chamber 110 having a length 116 and a diameter 114, the plunger 122 having a head 124; a pusher 202 in sliding engagement with the base 134, the pusher 202 structured and arranged to contact the head 124 of the plunger 122; a puller 200 in sliding engagement with the base 134; an adjustable driver 208 structured and arranged to provide a constant force between the pusher 202 and the puller 200, the adjustable driver 208 provided by a spring 210 connected between the pusher 202 and the puller 200 and providing a first force and an adjustable braker 212 structured and arranged to provide an adjustable braking force to reduce the first force, the constant force adjustably selected by adjusting the adjustable braking force applied to reduce the first force; whereby slidably moving the puller 200 distally when the syringe 102 is seated in the base 134 causing the pusher 202 to contact and exert a substantially constant force on the head 124 of the plunger 122, the constant force sufficient to move the plunger 122 of the syringe 102 and dispense a liquid from the chamber 110 at a desired flow rate; and a cover 136 connected to the base 134, a linkage coupled between the cover 136 and the puller 200 and structured and arranged to translate the opening of the cover 136 to the movement of the puller 200 and pusher 202 towards the proximal end 132, and the closing of the cover 136 when the syringe 102 has been disposed engages the pusher 202 against the plunger 122 and moves the puller 200 towards the distal end 132 and engages the adjustable driver 208.

Yet another embodiment of a CFPDSS 100 may be summarized as: an expandable base 134 having a proximal end 132 and a distal end 132, the base 134 comprising: a first base 134 section and a second base 134 section, wherein the first base 134 section is in sliding engagement with the second base 134 section such that the first base 134 section and the second base 134 section are slidably movable relative to each other between a compacted position and an expanded position, wherein the base 134 in the expanded position is adapted to seat a syringe 102 having a plunger 122 slidably disposed within a chamber 110 having an outlet 108, the chamber 110 having a length 116 and a diameter 114, the plunger 122 having a head 124; a pusher 202 in sliding engagement with the base 134, wherein the pusher 202 is dimensioned to contact the head 124 of the plunger 122; a puller 200 in sliding engagement with the base 134; an adjustable driver 208 structured and arranged to provide a constant force between the pusher 202 and the puller 200, the adjustable driver 208 provided by a spring 210 connected between the pusher 202 and the puller 200 and providing a first force and an adjustable braker 212 structured and arranged to provide an adjustable braking force to reduce the first force, the constant force adjustably selected by adjusting the adjustable braking force applied to reduce the first force; whereby slidably moving the puller 200 distally when the syringe 102 is seated in the base 134 causing the pusher 202 to contact and exert a substantially constant force on the head 124 of the plunger 122, the constant force sufficient to move the plunger 122 of the syringe 102 and dispense a liquid from the chamber 110 at a desired flow rate; and an expandable cover 136 pivotally connected to the proximal end 132 of the base 134, a linkage pivotally coupled between the cover 136 and the puller 200 and structured and arranged to translate the opening of the cover 136 to the movement of the puller 200 and pusher 202 towards the proximal end 132, and the closing of the cover 136 when the syringe 102 has been disposed engages the pusher 202 against the plunger 122 and moves the puller 200 towards the distal end 132 and engages the adjustable driver 208.

And further still, yet another embodiment of a CFPDSS 100 may be summarized as: a base 134 having a proximal end 132 and a distal end 132, the base 134 structured and arranged to receive a syringe 102 having a plunger 122 slidably disposed within a chamber 110 having an outlet 108, the chamber 110 having a length 116 and a diameter 114, the plunger 122 having a head 124; a pusher 202 in sliding engagement with the base 134, the pusher 202 structured and arranged to contact the head 124 of the plunger 122; a puller 200 in sliding engagement with the base 134; an adjustable driver 208 structured and arranged to provide a constant force between the pusher 202 and the puller 200, the adjustable driver 208 provided by a spring 210 connected between the pusher 202 and the puller 200 and providing a first force and an adjustable braker 212 structured and arranged to provide an adjustable braking force to reduce the first force, the constant force adjustably selected by adjusting the adjustable braking force applied to reduce the first force; whereby slidably moving the puller 200 distally when the syringe 102 is seated in the base 134 causing the pusher 202 to contact and exert a substantially constant force on the head 124 of the plunger 122, the constant force sufficient to move the plunger 122 of the syringe 102 and dispense a liquid from the chamber 110 at a desired flow rate; and a cover 136 pivotally connected to the proximal end 132 of the base 134, a linkage pivotally coupled between the cover 136 and the puller 200 and structured and arranged to translate the opening of the cover 136 to the movement of the puller 200 and pusher 202 towards the proximal end 132, and the closing of the cover 136 when the syringe 102 has been disposed engages the pusher 202 against the plunger 122 and moves the puller 200 towards the distal end 132 and tensions spring 210 to apply the constant force to the plunger 122 of the syringe 102.

Having described embodiments of the CFPDSS 100, other embodiments relating to at least one method of using CFPDSS 100 for providing infusion therapy into subcutaneous tissues will now be discussed. It will be appreciated that the described methods need not be performed in the order in which they are herein described, but that these descriptions are merely exemplary of methods for providing and using CFPDSS 100.

KORU Medical Systems, Inc. of Mahwah, New Jersey, is and has been a pioneer in infusion pump technology, needle set technology and flow rate control by means of specifically engineered flow control tubing. Indeed, KORU has realized that different flow rates may be provided by working with different flow combinations of flow control tubing, such as those systems and methods set forth in U.S. Pat. No. 10,420,886 entitled MULTI-FLOW UNIVERSAL TUBING SET, incorporated herein by reference, and U.S. Pat. No. 10,709,839 entitled PRECISION VARIABLE FLOW RATE INFUSION SYSTEM AND METHOD, incorporated herein by reference.

Further, KORU has developed advantageous infusion systems permitting high flow at low pressure as set forth in U.S. application Ser. No. 17/729,914 published as US 2022/0265923 entitled HIGH FLOW AT LOW PRESSURE INFUSION SYSTEM, incorporated herein by reference. Further still, for at least one embodiment the needle(s) used for the infusion therapy may be is the snap in needle structure shown and described in U.S. patent application Ser. No. 18/216,342 entitled SYSTEM AND METHOD FOR BUTTERFLY NEEDLE ASSEMBLY, incorporated herein by reference.

Moreover, it will be understood and appreciated that CFPDSS 100 may serve as the constant force pump for infusion therapy provided to a patient incorporating one or more of the above identified technologies. In addition, as noted above, for varying embodiments, where the syringe is coupled to a lure, such as the flared luer as set forth in U.S. Pat. No. 10,500,389 entitled, SYSTEM AND METHOD FOR FLARED LUER CONNECTOR FOR MEDICAL TUBING or a tapered luer as set forth in U.S. Provisional Application 63/616,368 entitled, SYSTEM AND METHOD FOR A TAPERED LURE CONNECTOR FOR MEDICAL TUBING, the selector 222, and more specifically the seat 224 of the selector may be structured and arranged as an accepting base for such a tapered or flared luer.

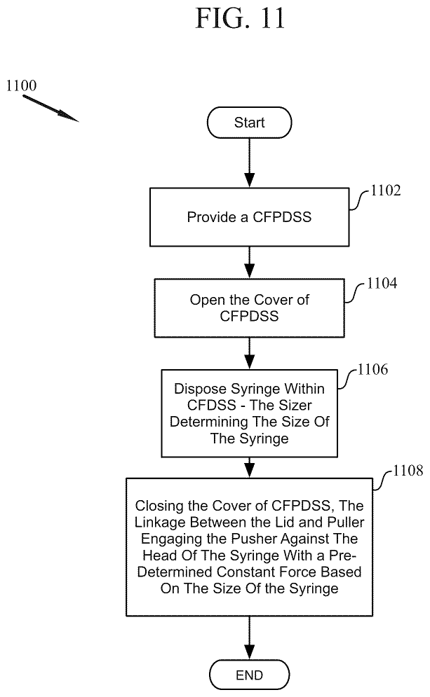

Turning to FIG. 11, presented is a flow diagram for at least one method 1100 of using CFPDSS 100 for infusion therapy. In general, method 1100 commences with providing a CFPDSS 100, block 1102.

The cover 136 of CFPDSS 100 it then opened, block 1104. A selected syringe is then disposed within CFPDSS 100. As discussed above, CFPDSS 100 is structured and arranged to use a sizer 220 to determine the size of the disposed syringe 102 and mechanically engage and adjustable driver 208 to provide a pre-determined constant force appropriate for the disposed syringe, block 1106.

To fully engage CFPDSS 100 with the pre-determined constant force, the cover 136 of CFPDSS 100 is then closed, and the infusion process is initiated to the patient, block 1108. As CFPDSS 100 is advantageously structured and arranged to determine the size of the syringe, and then the pre-determined constant force for the syringe based on the determined size, it will be understood and appreciated that the same CFPDSS 100 may be used for the infusion therapy treatment of different sized syringes without adverse effects from improper force applied for the administration. Moreover, a single CFPDSS 100 is advantageously capable of self-determining different syringe sizes and automatically selecting the pre-determined constant force appropriate for the administration of the medicate within a syringe of a determined size.

Changes may be made in the above methods, systems and structures without departing from the scope hereof. It should thus be noted that the matter contained in the above description and/or shown in the accompanying drawings should be interpreted as illustrative and not in a limiting sense. Indeed, many other embodiments are feasible and possible, as will be evident to one of ordinary skill in the art. The claims that follow are not limited by or to the embodiments discussed herein, but are limited solely by their terms and the Doctrine of Equivalents.

What is claimed:

1. A constant force syringe pump assembly accommodating syringes of different sizes comprising:
a base having a proximal end and a distal end, the base structured and arranged to receive a syringe having a plunger slidably disposed within a chamber having an outlet, the chamber having a length and a diameter, the plunger having a head;
a pusher in sliding engagement with the base, the pusher structured and arranged to contact the head of the plunger;
a puller in sliding engagement with the base;
an adjustable driver structured and arranged to provide a constant force between the pusher and the puller, the adjustable driver provided by at least one spring connected between the pusher and the puller and providing a first force and an adjustable braker structured and arranged to provide an adjustable braking force to reduce the first force, the constant force adjustably selected by adjusting the adjustable braking force applied to reduce the first force;
whereby slidably moving the puller distally when the syringe is seated in the base causes the pusher to contact and exert a constant force on the head of the plunger, the constant force sufficient to move the plunger of the syringe and dispense a liquid from the chamber at a desired flow rate; and
a cover connected to the base, a linkage coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

2. The constant force syringe pump of claim 1, wherein the adjustable braker is associated with the at least one spring, the adjustable braker providing the adjustable braking force to the at least one spring.

3. The constant force syringe pump of claim 1, wherein the adjustable braker is associated with the pusher, the adjustable braker providing the adjustable braking force to the pusher.

4. The constant force syringe pump of claim 1, wherein the adjustable braking force is selected by selecting a pre-defined setting for the adjustable breaker.

5. The constant force syringe pump of claim 1, wherein the adjustable braking force is user modifiable to increase or decrease the constant force on the head of the plunger.

6. The constant force syringe pump of claim 1, wherein the constant force pump has a flow rate indicator, the braking force selected by adjusting the adjustable braker to a desired flow rate shown by the flow rate indicator.

7. The constant force syringe pump of claim 1, wherein the adjustable braking force is selected by a sizer provided at least in part by at least one controller associated with the adjustable braker and a selector, the selector being movable between a first position to a second position to establish the size of the chamber, the sliding of the selector from the first position to the second position causing controller to adjust the adjustable braker to adjust the braking force to the at least one spring to provide the constant force specific for the size of the chamber.

8. The constant force syringe pump of claim 1, wherein the cover is connected to the base by a unidirectional friction hinge providing a first resistance in a closing operation and a second resistance in an opening operation, the second resistance greater than the first.

9. The constant force syringe pump of claim 1, wherein the pump has a safety clasp, automatically binding the base and cover as the cover is closed, and requiring a user to activate a release to open the cover.

10. The constant force syringe pump of claim 1, wherein the constant force provided by the adjustable driver is between about 1 to 25 pounds of force.

11. The constant force syringe pump of claim 1, wherein the pump is structured and arranged to accommodate a syringe having a volume of at least 5 milliliters and no more than 120 milliliters.

12. The constant force syringe pump of claim 1, wherein the adjustable braker is a friction braker.

13. The constant force syringe pump of claim 1, wherein the adjustable braker is a pneumatic brake.

14. The constant force syringe pump of claim 1, wherein the adjustable braker is a disc brake.

15. The constant force syringe pump of claim 1, wherein the adjustable braker is a dampener.

16. The constant force syringe pump of claim 1, wherein the pump has an adjustable carrying strap.

17. A constant force syringe pump assembly accommodating syringes of different sizes comprising:
an expandable base having a proximal end and a distal end, the base comprising: a first base section and a second base section, wherein the first base section is in sliding engagement with the second base section such that the first base section and the second base section are slidably movable relative to each other between a compacted position and an expanded position, wherein the base in the expanded position is adapted to seat a syringe having a plunger slidably disposed within a chamber having an outlet, the chamber having a length and a diameter, the plunger having a head;
a pusher in sliding engagement with the base, wherein the pusher is dimensioned to contact the head of the plunger;
a puller in sliding engagement with the base;
an adjustable driver structured and arranged to provide a constant force between the pusher and the puller, the adjustable driver provided by a at least one spring connected between the pusher and the puller and providing a first force and an adjustable braker structured and arranged to provide an adjustable braking force to reduce the first force, the constant force adjustably selected by adjusting the adjustable braking force applied to reduce the first force;
whereby slidably moving the puller distally when the syringe is seated in the base causes the pusher to contact and exert a constant force on the head of the plunger, the constant force sufficient to move the plunger of the syringe and dispense a liquid from the chamber at a desired flow rate; and
an expandable cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and engages the adjustable driver.

18. The constant force syringe pump of claim 17, wherein the adjustable braker is associated with the at least one spring, the adjustable braker providing the adjustable braking force to the at least one spring.

19. The constant force syringe pump of claim 17, wherein the adjustable braker is associated with the pusher, the adjustable braker providing the adjustable braking force to the pusher.

20. The constant force syringe pump of claim 17, wherein the adjustable braking force is selected by selecting a pre-defined setting for the adjustable breaker.

21. The constant force syringe pump of claim 17, wherein the adjustable braking force is user modifiable to increase or decrease the constant force on the head of the plunger.

22. The constant force syringe pump of claim 17, wherein the constant force provided by the adjustable driver is between about 1 to 25 pounds of force.

23. The constant force syringe pump of claim 17, wherein the pump is structured and arranged to accommodate a syringe having a volume of at least 5 milliliters and no more than 120 milliliters.

24. The constant force syringe pump of claim 17, wherein the constant force pump has a flow rate indicator, the braking force selected by adjusting the adjustable braker to a desired flow rate shown by the flow rate indicator.

25. The constant force syringe pump of claim 17, wherein the adjustable braking force is selected by a sizer provided at least in part by at least one controller associated with the adjustable braker and a selector, the selector being movable between a first position to a second position to establish the size of the chamber, the sliding of the selector from the first position to the second position causing controller to adjust the adjustable braker to adjust the braking force to the at least one spring to provide the constant force specific for the size of the chamber.

26. The constant force syringe pump of claim 17, wherein the cover is connected to the base by a unidirectional friction hinge providing a first resistance in a closing operation and a second resistance in an opening operation, the second resistance greater than the first.

27. A constant force syringe pump assembly accommodating syringes of different sizes comprising:
a base having a proximal end and a distal end, the base structured and arranged to receive a syringe having a plunger slidably disposed within a chamber having an outlet, the chamber having a length and a diameter, the plunger having a head;
a pusher in sliding engagement with the base, the pusher structured and arranged to contact the head of the plunger;
a puller in sliding engagement with the base;
an adjustable driver structured and arranged to provide a constant force between the pusher and the puller, the adjustable driver provided by a at least one spring connected between the pusher and the puller and providing a first force and an adjustable braker structured and arranged to provide an adjustable braking force to reduce the first force, the constant force adjustably selected by adjusting the adjustable braking force applied to reduce the first force;
whereby slidably moving the puller distally when the syringe is seated in the base causes the pusher to contact and exert a constant force on the head of the plunger, the constant force sufficient to move the plunger of the syringe and dispense a liquid from the chamber at a desired flow rate; and
a cover pivotally connected to the proximal end of the base, a linkage pivotally coupled between the cover and the puller and structured and arranged to translate the opening of the cover to the movement of the puller and pusher towards the proximal end, and the closing of the cover when the syringe has been disposed engages the pusher against the plunger and moves the puller towards the distal end and tensions the at least one spring to apply the constant force to the plunger of the syringe.

28. The constant force syringe pump of claim 27, wherein the adjustable braker is associated with the at least one spring, the adjustable braker providing the adjustable braking force to the at least one spring.

29. The constant force syringe pump of claim 27, wherein the adjustable braker is associated with the pusher, the adjustable braker providing the adjustable braking force to the pusher.

30. The constant force syringe pump of claim 27, wherein the adjustable braking force is selected by selecting a pre-defined setting for the adjustable breaker.

31. The constant force syringe pump of claim 27, wherein the adjustable braking force is user modifiable to increase or decrease the constant force on the head of the plunger.

32. The constant force syringe pump of claim 27, wherein the constant force provided by the adjustable driver is between about 1 to 25 pounds of force.

33. The constant force syringe pump of claim 27, wherein the pump is structured and arranged to accommodate a syringe having a volume of at least 5 milliliters and no more than 120 milliliters.

34. The constant force syringe pump of claim 27, wherein the constant force pump has a flow rate indicator, the braking force selected by adjusting the adjustable braker to a desired flow rate shown by the flow rate indicator.

35. The constant force syringe pump of claim 27, wherein the adjustable braking force is selected by a sizer provided at least in part by at least one controller associated with the adjustable braker and a selector, the selector being movable between a first position to a second position to establish the size of the chamber, the sliding of the selector from the first position to the second position causing controller to adjust the adjustable braker to adjust the braking force to the at least one spring to provide the constant force specific for the size of the chamber.

36. The constant force syringe pump of claim 27, wherein the cover is connected to the base by a unidirectional friction hinge providing a first resistance in a closing operation and a second resistance in an opening operation, the second resistance greater than the first.
